SEQUENCE LISTING (1 sequence)

SEQ ID NO: 1 - protein, 147 aa, Rattus norvegicus
  MISC_FEATURE (93)..(93): X is U
  MISC_FEATURE (95)..(95): X is U
MKSLYLIFGLWILLACFQSGEGVRGPRRQHNPRRQQDPSTLPHYLGLQPDPNLGGQIGVTITIPLNLQPPRVLVNLPGFITGPPLVVQGTTEXQXQWQLTAPDPTPLSNPPTQLLSTEQANTKTDAKISNTTATTQNSTDIFEGGGK

(19) United States
(12) Patent Application Publication
(10) Pub. No.: US 2019/0015675 A1
(43) Pub. Date: Jan. 17, 2019

(54) NEUROMODULATION APPARATUS

(71) Applicant: GALVANI BIOELECTRONICS LIMITED, Brentford Middlesex (GB)

(72) Inventors: Alessandra GIAROLA, Stevenage, Hertfordshire (GB); Arun SRIDHAR, Stevenage, Hertfordshire (GB); Nicolas WISNIACKI, Stevenage, Hertfordshire (GB)

(73) Assignee: GALVANI BIOELECTRONICS LIMITED, Brentford Middlesex (GB)

(21) Appl. No.: 16/080,143

(22) PCT Filed: Feb. 27, 2017
(86) PCT No.: PCT/IB2017/051145

§ 371 Date: Aug. 27, 2018

Related U.S. Application Data
(60) Provisional application No. 62/301,208, filed on Feb. 29, 2016.

(51) Int. Cl. A61N 2/00; A61N 5/06; A61N 7/00; A61F 7/00; A61H 7/00

(52) U.S. Cl. CPC A61N 2/006; A61N 5/0622; A61N 1/3606; A61F 7/00; A61H 7/00; A61N 7/00

(57) ABSTRACT

The present disclosure provides an apparatus or system and methods for treating xerostomia or Sjögren's Syndrome in a subject.

### BACKGROUND

Xerostomia is a condition defined as dry mouth resulting from reduced or absent saliva flow. It is a common side-effect of certain medications and treatments, notably cancer chemotherapeutic drugs and radiation therapy. It is also caused by medications such as antihistamines, antidepressants, anticholinergics, anorexiants, antihypertensives, antipsychotics, anti-Parkinson agents, diuretics and sedatives. Xerostomia is also a symptom associated with a variety of diseases including rheumatic disorders such as rheumatoid arthritis, systemic lupus erythematosus and scleroderma, diabetes mellitus, cystic fibrosis, cytomegalovirus and other herpes viruses, hepatitis C, ectodermal dysplasia, chronic pancreatitis, and celiac disease among others.

The most common disease causing xerostomia is Sjögren's Syndrome (SS). SS is a chronic, slowly progressive autoimmune disease that occurs predominantly in postmenopausal women. Patients with SS experience damage to the salivary and lacrimal glands caused by lymphocytic infiltration, and consequently present with symptoms including oral and ocular dryness (xerostomia and xerophthalmia, respectively). These “sicca” symptoms significantly impact the patient's perception of health-related quality of life, and since there is no cure for this disease, the alleviation of symptoms plays an important role in patient management. To restore the salivary output to normal levels, local salivary stimulations (e.g. chewing gums, tablets, lozenges) and cholinergic agonists are currently used (Gonzalez et al., 2014. Oral manifestations and their treatment in Sjögren's Syndrome. Oral Diseases 20:153-161).

Electrical stimulation within the oral cavity has been described as a non-pharmacological means of treating xerostomia. Devices such as the Salitron (Biosonics®, PA), Saliwell (GenNarino®) and Saliwell Crown®, have been described as neuro-electro-stimulators for the treating of xerostomia, see for example Lafaurie et al. 2009. Biotechnological advances in neuro-electro-stimulation for the treatment of hyposalivation and xerostomia. Med Oral Patol Oral Circ Bucal. 14(2):E76-E80. Acupuncture-like transcutaneous electrical nerve stimulation (ALIENS) has also been described for the treatment of xerostomia, particularly xerostomia caused by radiotherapy in cancer patients (Wong et al., 2012. Phase 2 results from Radiation Therapy Oncology Group Study 0537: A phase 2/3 study comparing acupuncture-like transcutaneous electrical nerve stimulation versus pilocarpine in treating early radiation-induced xerostomia. Cancer. 118(17):4244-4252).

Despite the treatments available, there is a need to develop alternative treatments to alleviate xerostomia, particularly xerostomia in patients with Sjögren's Syndrome.

### SUMMARY OF INVENTION

The present disclosure provides systems and methods for the alleviation of xerostomia (e.g., in patients with Sjögren's Syndrome). The present disclosure also provides systems and methods for the alleviation of Sjögren's Syndrome.

In one aspect, the present invention provides an apparatus or system for stimulating neural activity in a superior cervical ganglion (SCG), for example the preganglionic and/or postganglionic neurons of a SCG, of a subject, the apparatus comprising: one or more neural interfacing elements (e.g., transducers), each configured to apply a signal to said SCG of the subject; and a controller operably coupled to the one or more neural interfacing elements. The controller controls the signal to be applied by each of the one or more neural interfacing elements, such that the signal stimulates the neural activity of said SCG to produce a physiological response in the subject. Preferably the response is an increase in saliva production and/or an increase in anti-inflammatory peptides in the saliva of the subject. Such an apparatus or system is an apparatus or system for treating xerostomia or Sjögren's Syndrome in a subject.

In another aspect, the present invention provides a method of treating xerostomia, particularly xerostomia associated with Sjögren's Syndrome, in a subject. In a further aspect, the invention provides a method of treating Sjögren's Syndrome. In such aspects, the methods comprise: (i) implanting in the subject an apparatus as described above; (ii) positioning at least one transducer of the apparatus in signalling contact with a superior cervical ganglion (SCG) of a subject, for example preganglionic and/or postganglionic neurons of the SCG; (iii) activating the apparatus.

In another aspect, the present invention provides a method of treating xerostomia, particularly xerostomia associated with Sjögren's Syndrome, in a subject. In a further aspect, the invention provides a method of treating Sjögren's Syndrome in a subject. In such aspects the method comprises applying a signal to a superior cervical ganglion of said subject, for example a preganglionic and/or postganglionic neuron of the SCG, to stimulate neural activity in said SCG in the subject.

In another aspect, the present invention provides a saliva substitute or saliva stimulant for use in a method of treating xerostomia, particularly xerostomia associated with Sjögren's Syndrome, in a subject. In a further aspect, the invention provides a saliva substitute or saliva stimulant for use in a method of treating Sjögren's Syndrome in a subject. In such aspects, the method comprises: (i) applying a signal to a superior cervical ganglion, for example a preganglionic and/or a postganglionic neuron of a SCG, of said subject to stimulate neural activity in said SCG; and (ii) administering the saliva substitute or saliva stimulant to the subject.

In another aspect, the invention provides a saliva substitute or saliva stimulant for use in a method of treating xerostomia in a subject, for example xerostomia associated with Sjögren's Syndrome. In a further aspect, the invention provides a saliva substitute or saliva stimulant for use in a method of treating Sjögren's Syndrome in a subject. In such aspects, the method comprises administering the saliva substitute or saliva stimulant to the subject, the subject having an apparatus according to any one of claims 1-17 implanted such that the neural interfacing element is positioned in signalling contact with a superior cervical ganglion (for example a preganglionic and/or post-ganglionic neuron of a SCG) of the subject.

In another aspect, the present invention provides a neuromodulatory electrical waveform for use in treating xerostomia, particularly xerostomia associated with Sjögren's Syndrome, in a subject, or for treating Sjögren's Syndrome in a subject, wherein the waveform is a direct current (DC) waveform having a frequency of 1-1000 Hz, such that, when applied to a superior cervical ganglion, for example a preganglionic and/or post-ganglionic neuron of a SCG, of the subject, the waveform stimulates neural signalling in the neurons.

In another aspect, the present invention provides use of a neuromodulation apparatus for treating xerostomia, particularly xerostomia associated with Sjögren's Syndrome, in a subject. In a further aspect the invention provides use of a neuromodulation apparatus for treating Sjögren's Syndrome in a subject. In such aspects, the use is by stimulating neural activity in a superior cervical ganglion of the subject, for example preganglionic and/or postganglionic neurons of a SCG.

In a preferred embodiment of all aspects of the invention, the subject is a human, such as a human patient suffering from Sjögren's Syndrome.

### BRIEF DESCRIPTION OF DRAWINGS

FIG. 1: Sympathetic and parasympathetic innervations of the submandibular gland.

FIG. 2: Schematic drawings showing how apparatuses, devices and methods according to the invention can be put into effect.

FIG. 3: (A) Schematic of laser Doppler pulses. 1=baseline; 2=peak response; 3=total pulsatile units; AUC=total response. (B) Laser Doppler signals from mouse SMG.

FIG. 4: Submandibular gland (SMG) laser Doppler pulse following back paw pinch (6s) showing increase in blood flow in SMG during pinch. (A) 6s pinch results in increase in blood flow followed by return to normal blood flow. (B) 8s and 12s pinch. 12s pinch results in increase in blood flow that is persistent.

FIG. 5: SMG laser Doppler pulse during airway occlusion. Airway occlusion initially causes a marked increase in blood flow in the SMG as can be seen during times 5-20 seconds. The increases in flow are especially marked during pronounced inspiratory effort.

FIG. 6: (A) Electrical stimulation (0.8 mA, 5 Hz) of the right cervical sympathetic chain (CSC) increases blood flow in the right SMG. (B) Transection of the ipsilateral (right) internal carotid nerve (ICN) and external carotid nerve (ECN) markedly diminishes the response elicited by electrical stimulation of the right CSC (0.8 mA, 5 Hz).

FIG. 7: (A) Example sequence of SMR1 (SEQ ID NO: 1) showing positions of anti-inflammatory peptides derived therefrom and also showing epitopes of anti-SMR1 antibodies 216 and 219 produced according to Morris et al., 2009, Am J Physiol Cell Physiol. 296: C514-C524. (B) Western blots showing detection of SMR1 from rat SMG (right panels) and comparator blot from Morris et al., 2009, ibid, showing equivalent position of SMR1 (left panel).

FIG. 8: Western blot showing levels of SMR1 in collected saliva from rats #1, #2 and #3 prior to stimulation, following electrical stimulation, following isoproterenol administration, and following isoproterenol and electrical stimulation. (A) rats #1 and #2; (B) rats #2 and #3, plus relative amounts of SMR1 following each treatment, normalised to levels of β-actin.

FIG. 9: (Top) Volume of saliva collected (μl) and (Bottom) the total content of protein in the collected saliva (mg/ml). Numbers 1, 5 and 9 are prestimulation samples; 2, 6 and 10 post-stimulation, 3, 7 and 11 post-isoproterenol; 4, 8 and 12 post-stimulation and isoproterenol for rats #1, #2, and #3, respectively.

FIG. 10: (Top) Volume of saliva collected (μl) and (Bottom) total content of protein in the collected saliva (mg/ml). 1. 30 min collection of saliva; 2. Stimulation of Left SCG—15 min stimulation and collection for 15 min+5 min; 3. 15-20 min collection; 4. Stimulation of left SCG—15 min stimulation and collection for 15+5 min; 5. 15-20 min collection; 6. Stimulation 3 of Right SCG—15 min stimulation and collection for 15 min+5 min; 7. 15-20 minute collection; 8. Stimulation 4 of Right SCG—15 min stimulation and collection for 15 min+5 min.

FIG. 11: Western blots of SMR1 levels in saliva collected during stimulations 1-4 of FIG. 10. Equal protein was loaded to each lane.

### DETAILED DESCRIPTION

Salivary gland secretion is regulated by the autonomic nervous system, with innervation provided by parasympathetic and sympathetic fibres. The major salivary glands are the parotid, submandibular, and sublingual glands. The submandibular glands are located beneath the floor of the mouth and produce a mixed serous and mucous secretion. This secretion contributes about 70% of the salivary volume under unstimulated conditions.

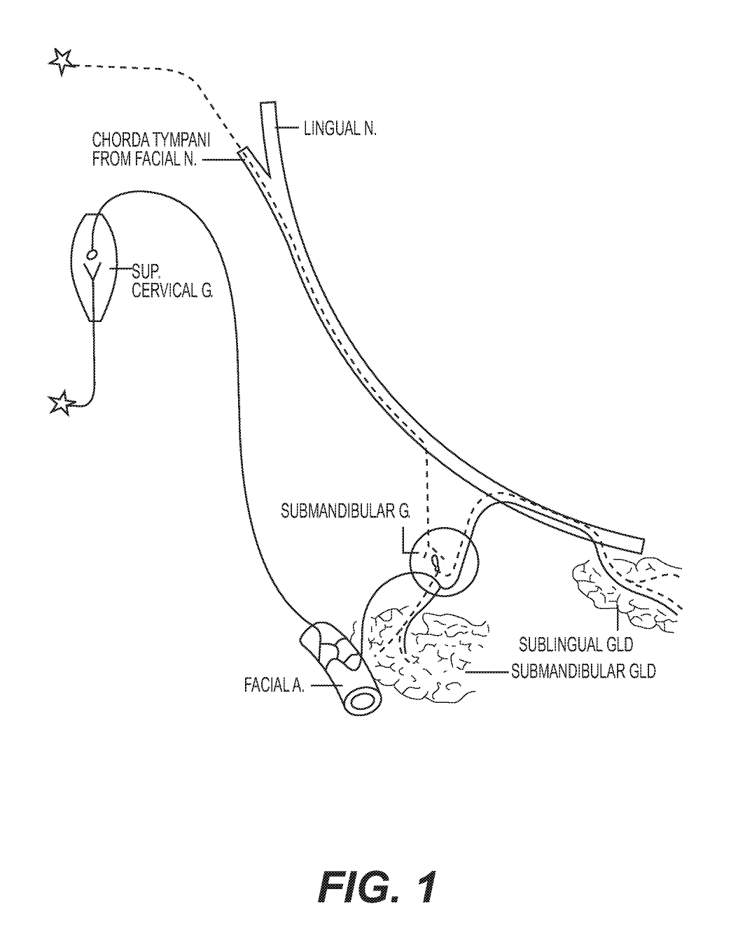

The submandibular glands receive sympathetic postganglionic innervations from the Superior Cervical Ganglia (SCG) (see FIG. 1). The SCG are bilateral structures residing in close proximity to the carotid body at the trifurcation of the common carotid artery into the internal and external carotid arteries and the occipital artery. The SCG and the submandibular glands, together, form a neuroendocrine axis called the cervical sympathetic trunk submandibular gland (CST-SMG) axis (also known as the cervical sympathetic chain SMG axis, or CSC-SMG). Preganglionic neurons of the SMG form the cervical sympathetic trunk or chain (CST/CSC), with the SCG containing the cell bodies of postganglionic neurons of the SCG innervating the SMG. Given the importance of exocrine and endocrine secretions from the salivary glands, notably the submandibular glands, this neuroendocrine axis plays a fundamental role in systemic homeostasis.

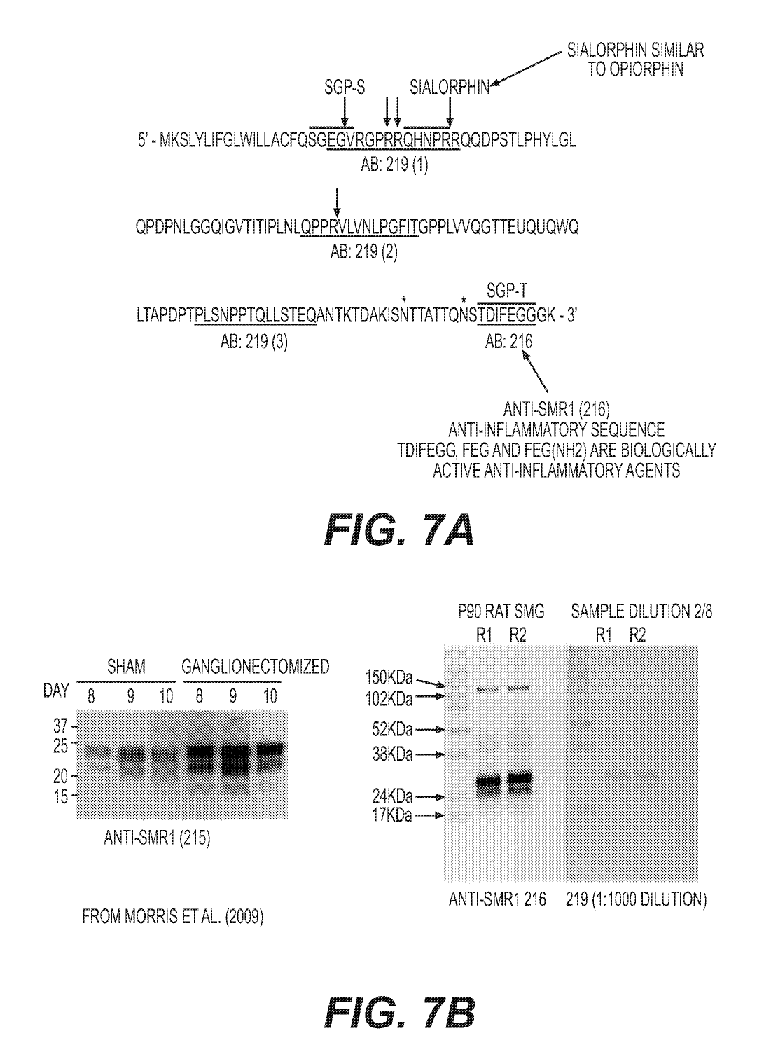

The SMG is an important source of systemically active immunoregulatory and anti-inflammatory factors whose release is actively controlled by the autonomic nervous system, and in particular the SCG [Mathison et al., 2012, Biestock J (ed): Allergy and the Nervous System. Chem Immunol Allergy 98: 176-195, incorporated herein by reference]. One biological end component of the CSC-SMG axis is the synthesis, processing and release of submandibular rat-1 protein (SMR1), a pro-hormone that generates several different peptides (an example amino acid sequence of SMR1 is given in FIG. 7A (SEQ ID NO: 1); other recognised sequences of SMR1 are provided in Rosinski-Chupin I & Rougeon F. DNA Cell Biol. 1990 October; 9(8):553-9, and UniProt database entry P13432, each of which is incorporated herein by reference). These peptides include a tripeptide fragment phenylalanine-glutamic acid-glycine (FEG) and its metabolically stable isomer feG, which are potent inhibitors of allergy and asthma (IgE-mediated allergic reactions) and several non IgE-mediated inflammatory states [Dery et al., 2001, Int Arch Allergy Immunol. 124: 201-204; Dery et al., 2004, Eur J Immunol. 34: 3315-3325; Morris et al., 2009, Am J Physiol Cell Physiol. 296: C514-C524; Mathison et al., 2009, Open Inflam J. 2: 9-21; Mathison et al 2010 J Inflamm. 7: 49; Mathison et al. 2012, supra; Laurent et al., 2015, Am J Physiol Regul Integr Comp Physiol. 308: R569-R575, each of which is incorporated herein by reference]. Another key amino acid sequence derived from SMR1 is QHNPR (sialorphin), located near the NH2 terminus of SMR1. This peptide has analgesic activity [Rougerot et al., 2003, Proc Natl Acad Sci USA 100:8549-8554, incorporated herein by reference]. Calcium-binding protein spermatid-specific 1 (CABS1) is human homolog of SMR1. CABS1 has been found in salivary glands and ducts in humans, has a similar amino acid sequence to SMR1 and may have similar biological activity and functional roles as the SMR1 proteins and peptides [Laurent et al., 2015, supra]. Sialorphin is similar to the opiorphin peptide present in human saliva.

The present disclosure concerns an apparatus or system and methods for stimulating the SCG to increase saliva production (e.g., volume or secretion) and/or to improve the quality of saliva, for example by the production of increased levels of anti-inflammatory peptides in the saliva. Such an apparatus or system and methods are useful for the treatment of xerostomia, such as that associated with Sjögren's Syndrome (SS).

The terms as used herein are given their conventional definition in the art as understood by the skilled person, unless otherwise defined below. In the case of any inconsistency or doubt, the definition as provided herein should take precedence.

As used herein, application of a signal may equate to the transfer of energy in a suitable form to carry out the intended effect of the signal. That is, application of a signal to neurons, a nerve or nerves may equate to the transfer of energy to (or from) the neurons or nerve(s) to carry out the intended effect. For example, the energy transferred may be electrical, mechanical (including acoustic, such as ultrasound), electromagnetic (e.g. optical), magnetic or thermal energy. It is noted that application of a signal as used herein does not include a pharmaceutical intervention.

As used herein, a “neural interfacing element” or “transducer” is taken to mean any element of applying a signal to the neurons or nerve or plexus, for example an electrode, diode, Peltier element or ultrasound transducer.

As used herein, a “non-destructive signal” is a signal as defined above that, when applied, does not irreversibly damage the underlying neural signal conduction ability. That is, application of a non-destructive signal maintains the ability of the neurons, nerve or nerves (or fibres thereof) to conduct action potentials when application of the signal ceases, even if that conduction is in practice inhibited or blocked as a result of application of the non-destructive signal. Ablation and cauterisation of at least part of the nerve are examples of destructive signals.

As used herein, “neural activity” of a neuron or nerve is taken to mean the signalling activity of the neuron or nerve, for example the amplitude, frequency and/or pattern of action potentials in the neuron or nerve.

Stimulation of neural activity as used herein may be an increase in the total signalling activity of the whole nerve, or that the total signalling activity of a subset of neurons or nerve fibres of the nerve is increased, compared to baseline neural activity in that part of the nerve.

Neural activity of a neuron or nerve may also be modulated to cause an alteration in the pattern of action potentials. It will be appreciated that the pattern of action potentials can be modulated without necessarily changing the overall frequency or amplitude. For example, modulation of the neural activity may be such that the pattern of action potentials is altered to more closely resemble a healthy state rather than a disease state.

Modulation of neural activity may comprise altering the neural activity in various other ways, for example increasing or inhibiting a particular part of the neural activity and/or stimulating new elements of activity, for example in particular intervals of time, in particular frequency bands, according to particular patterns and so forth. Such altering of neural activity may for example represent both increases and/or decreases with respect to the baseline activity.

Stimulation of neural activity may be selective for certain neurons or nerve fibres. As used herein, “selective stimulation” is used to mean that the signal preferentially increases the neural activity in a target class of neuron or nerve fibre compared to other classes of neuron or nerve fibre. Such a selective stimulation is characterised by an increase in the proportion of the target neurons or nerve fibres that show an increase of neural activity compared to the proportion of nerve fibres of other classes that show an increase of neural activity. Substantially selective stimulation is characterised by neural activity being increased in at least 70% of the target neurons or nerve fibres when neural activity is increased in no more than 10% of non-target neurons or nerve fibres.

Stimulation of the neural activity may be temporary. As used herein, “temporary” is taken to mean that the stimulated neural activity is not permanent. That is, the neural activity following cessation of the signal is substantially the same as the neural activity prior to the signal being applied i.e. prior to stimulation.

Stimulation of the neural activity may be persistent. As used herein, “persistent” is taken to mean that the stimulation of neural activity has a prolonged effect. That is, upon cessation of the signal, neural activity in the nerve remains substantially the same as when the signal was being applied i.e. the neural activity during and following stimulation is substantially the same.

Stimulation of the neural activity may be corrective. As used herein, “corrective” is taken to mean that the stimulated neural activity alters the neural activity towards the pattern of neural activity in a healthy individual. That is, upon cessation of the signal, neural activity in the neurons or nerve more closely resembles the pattern of action potentials in the neurons or nerve observed in a healthy subject than prior to stimulation, preferably substantially fully resembles the pattern of action potentials in the neurons or nerve observed in a healthy subject.

For example, application of the signal may result in a stimulation of neural activity, and upon cessation of the signal, the pattern of action potentials in the neurons or nerve resembles the pattern of action potentials observed in a healthy subject.

As used herein, the term “superior cervical ganglion” (abbreviated to SCG) refers to the largest of the three cervical ganglia, which form part of the sympathetic nervous system. The SCG is the only ganglion in the sympathetic nervous system that innervates the head and neck.

The SCG is innervated by the ciliospinal centre of the spinal cord within the intermediolateral column (thus, these are referred to as “preganglionic neurons” of the SCG) and synapse with neurons the cell bodies of which are located in the SCG and that project from the rostral end of the SCG and innervate target organs of the head (referred to as “postganglionic neurons” of the SCG).

The SCG contains the cell bodies of sympathetic neurons that project to a variety of structures in the brain (e.g., hypothalamus) in addition to the upper airways (e.g., larynx), tongue, and salivary glands (e.g., submandibular gland). The fibres of the postganglionic neurons exit the SCG via the internal carotid nerve and the external carotid nerve. Postganglionic neurons having fibres exiting the SCG are responsible for innervating many organs, glands and parts of the carotid system in the head and neck. There are different types of postganglionic neuron depending on the target site, ranging from low threshold to high threshold neurons. The neurons with a low threshold have a faster action potential firing rate, while the high threshold neurons have a slow firing rate. A distinction between postganglionic neuron types can be made via immunostaining wherein SCG neurons may be classified as either positive or negative for neuropeptide Y (NPY). Low threshold, NPY-negative neurons are secretomotor neurons, innervating salivary glands.

As used herein, the term “salivary glands” encompass the parotid, submandibular, and sublingual glands located in and around the mouth area.

As used herein, the “cervical sympathetic trunk submandibular gland axis” or “CST-SMG axis” (also referred to as the cervical sympathetic chain submandibular gland or CSC-SMG axis) is the term used to describe the neuroendocrine signalling axis formed as a result of the sympathetic signalling between the SCG (as defined above) and the submandibular gland(s) below the floor of the mouth. As used herein, neurons of the “CSC” or “CST” are used herein interchangeably herein to refer to preganglionic and/or postganglionic neurons of the SMG, such as those that innervate the SMG.

As used herein, “xerostomia” means persistent dryness of the mouth, typically caused by a lack of saliva production and/or reduced or absent saliva flow. Patients with Sjögren's Syndrome present with xerostomia, although xerostomia may be caused by other underlying diseases or conditions as detailed above. Xerostomia is also a side effect of certain medications and treatments including cancer chemotherapy and radiotherapy.

As used herein, “Sjögren's Syndrome” (abbreviated to SS) means the chronic, slowly progressive autoimmune disease that affects exocrine glands such as the lachrymal and salivary glands. Patients with SS present with a variety of signs and symptoms, the most frequent being ocular and oral dryness resulting from the damage to the lachrymal and salivary glands. Patients with SS may also present with systemic inflammation and/or local inflammation around the mouth (oral inflammation) and/or local inflammation around the eyes (ocular inflammation). The oral dryness or xerostomia that occurs in patients with SS is accompanied by a high degree of inflammation, which may contribute to the symptoms experienced by patients having this disease.

Xerostomia or oral dryness may be determined by a patient or a physician. Xerostomia or oral dryness can also be determined using salivary flow measurements (e.g. unstimulated whole saliometry (UWS), or stimulated whole saliometry (SWS). For example, xerostomia may be indicated by a UWS result of less than or equal to 0.1 ml/min, or by a SWS result of less than or equal to 0.3 ml/min. Xerostomia or oral dryness can also be determined by parotid scintigraphy, for example where the subject exhibits delayed uptake, reduced concentration and/or delayed excretion of a tracer. Xerostomia or oral dryness can also be determined by sialography, for example indicated by diffuse sialectasis (without obstruction of the major ducts).

Ocular dryness can be determined by ocular surface assessment by staining. For example, ocular dryness can be indicated by a van Bijsterveld score greater than or equal to 4 in both eyes, a grade greater than or equal to 2 on the Oxford scale, and/or a SICCA-OSS score of 3 or greater. Ocular dryness can also be determined by tear secretion assessment. For example, ocular dryness may be determined using the Schimer I test, where less than 5 mm of the paper after 5 min indicates ocular dryness, or by the Schimer II test, where less than 10 mm of the paper after 5 min indicates ocular dryness. Ocular dryness may also be determined by tear clearance assessment, such as a fluorescein clearance test. For example, a wetting length of less than 3 mm at the 10 min interval in a fluorescein clearance test can indicate ocular dryness. Ocular dryness can also be determined by assessing tear film stability. For example, a tear break up time of less than 10s and/or a non-invasive tear break up time of less than 40s can indicate ocular dryness.

Inflammation around the mouth (oral inflammation) as a result of SS can be determined by visual inspection by the subject or by a physician for example, by observance of swelling of one or more salivary glands. Oral inflammation can also be characterised by focal lymphocytic sialadenitis (FLS). FLS is an inflammation of one or more salivary glands (e.g. the parotid, SMG, sublingual gland, and minor salivary glands such as the labial salivary gland (LSG) and is detectable, for example, by haematoxylin and eosin staining of biopsy tissue. FLS can be characterised by the presence of foci of at least 50 mononuclear cells in a periductal or perivascular location. The mononuclear cell infiltrates in the lesions are predominantly T and B cells. Oral inflammation can also be characterised by epithelial cells surrounding lesions expressing increased levels of immuno-modulatory cytokines (e.g. TNFa, IL-6, IL-1, IL-18 and IL-22) (Kyriakidis et al., J Autoimmunity 2014 51:67-74; Boumba et al Br J Rheumatol. 1995 April; 34(4):326-33, each of which is incorporated herein by reference). Oral inflammation can also be characterised by ectopic expression in one or more salivary glands of lymphotoxins (LT) and/or lymphoid chemokines CXCL13, CCL19, CCL21 and CXCL12 (Bombardieri M and Pitzalis C. Curr Pharm Biotechnol. 2012 August; 13(10):1989-96, which is incorporated herein by reference). Levels of cytokines or chemokines can be determined by conventional techniques such as flow cytometry, qPCR and microarray.

Systemic inflammation associated with SS can be characterised by lymphocytic infiltrates in exocrine glands other than the salivary glands, and/or circulating auto-antibodies. Examples of such auto-antibodies include anti-Ro (e.g. anti-Ro52, anti-Ro60) antibodies, anti-La antibodies, anti-U1RNP, rheumatoid factor/anti-Fc antibodies, anti-cryoglobin, AMA (anti-mitochondrial antibodies), anti-CCP antibodies, anti-muscarinic 3 receptor antibodies, and anti-carbonic anhydrase antibodies. Such autoantibodies can be measured by, for example, Western blot or ELISA.

Other indications of systemic inflammation associated with SS include normochromic, normocytic anemia, leukopenia, lymphopenia, neutropenia, thrombocytopenia, raised erythrocyte sedimentation rate (ESR), hypergammaglobulinemia, raised serum IgG, raised levels of beta-2-microglobulin, free light chains of immunoglobulins, serum monoclonal band, anti-nuclear antibodies, and hypocomplementemia.

As used herein, the neural activity in the neurons of a healthy individual is that neural activity exhibited by a patient not suffering from xerostomia or Sjögren's Syndrome.

As used herein, an “improvement in a measurable physiological parameter” is taken to mean that for any given physiological parameter, an improvement is a change in the value of that parameter in the subject towards the normal value or normal range for that value i.e. towards the expected value in a healthy individual.

For an example, in a subject suffering from xerostomia, an improvement in a measurable parameter may be: a decrease in systemic sympathetic tone; an increase in salivary volume; an increase in the protein/peptide concentration of saliva (for example an increase in total protein concentration and/or an increase in an anti-inflammatory protein or peptide (e.g. SMR1, CABS1, FEG, feG, sialorphin, opiorphin or homologs thereof)); an increase in secretion from the salivary glands; and/or an increase in secretion from the submandibular gland(s).

For further example, in a subject suffering from Sjögren's Syndrome, an improvement in a measurable parameter may be: a decrease in systemic sympathetic tone; an increase in salivary volume; an increase in the protein/peptide concentration of saliva (for example an increase in total protein concentration and/or an increase in an anti-inflammatory protein or peptide (e.g. SMR1, CABS1, FEG, feG, sialorphin, opiorphin or homologs thereof)); an increase in secretion from the salivary glands; and/or an increase in secretion from the submandibular gland(s). In SS, an improvement in a measurable parameter may be a decrease in inflammation, for example oral and/or systemic inflammation, optionally accompanied by an improvement in one or more of the parameters recited above.

Techniques for measuring these parameters would be familiar to the skilled person. For example: systemic sympathetic tone can be determined by direct measurement of sympathetic nerve activity, by measurement of levels of urinary catecholamines, measurement of the sympatho-vagal balance via heart rate variability (lower heart rate variability being indicative of a decrease in sympathetic tone); salivary volume can be determined using salivary flow measurements (e.g. UWS, SWS); protein/peptide concentration can be determined by Western blot or ELISA; oral and/or systemic inflammation can be determined by measuring one or more of the indicators of said inflammation described above.

The physiological parameter may comprise an action potential or pattern of action potentials in neurons or a nerve of the subject. An improvement in such a parameter is characterised by the action potential or pattern of action potentials in the neurons or nerve more closely resembling that exhibited by a healthy individual than before the intervention.

As used herein, a physiological parameter is not affected by modulation of the neural activity if the parameter does not change as a result of the modulation from the average value of that parameter exhibited by the subject or patient when no intervention has been performed i.e. it does not depart from the baseline value for that parameter.

The skilled person will appreciate that the baseline for any neural activity or physiological parameter in an individual need not be a fixed or specific value, but rather can fluctuate within a normal range or may be an average value with associated error and confidence intervals. Suitable methods for determining baseline values would be well known to the skilled person.

As used herein, a measurable physiological parameter is detected in a subject when the value for that parameter exhibited by the subject at the time of detection is determined. A detector is any element able to make such a determination.

A “predefined threshold value” for a physiological parameter is the value for that parameter where that value or beyond must be exhibited by a subject or patient before the intervention is applied. For any given parameter, the threshold value may be a value indicative of xerostomia or Sjögren's Syndrome. Examples of such predefined threshold values include cervical sympathetic signalling lower than in a healthy individual, salivary production lower than in a healthy individual, salivary gland secretion lower than in a healthy individual; submandibular gland secretion lower than in a healthy individual, a peptide/protein concentration of the saliva lower than in a healthy individual. Appropriate values for any given parameter would be simply determined by the skilled person.

Such a threshold value for a given physiological parameter is exceeded if the value exhibited by the subject is beyond the threshold value that is, the exhibited value is a greater departure from the normal or healthy value for that parameter than the predefined threshold value.

Treatment of xerostomia as used herein may be prophylactic or therapeutic. Prophylactic treatment may be administered prior to the onset of symptoms i.e. oral dryness. Therapeutic treatment may be characterised by the alleviation of symptoms in a subject exhibiting oral dryness, for example a subject who has received medication resulting in xerostomia or a subject already suffering from a disease associated with xerostomia, for example Sjögren's Syndrome, or a subject previously diagnosed as having such a disease.

Treatment of Sjögren's Syndrome as used herein may be prophylactic or therapeutic. Prophylactic treatment may be administered prior to the onset of symptoms i.e. oral dryness. In a subject suffering from SS, therapeutic treatment may be characterised by the alleviation of oral dryness and/or a decrease in inflammation, for example oral and/or systemic inflammation. A decrease in oral and/or systemic inflammation can be characterised by a reduction in one or more of the indicators of said inflammation described above.

A “neuromodulation apparatus” or “neuromodulation device” (used interchangeably) as used herein is an apparatus configured to modulate the neural activity of neurons or a nerve. Neuromodulation apparatuses as described herein comprise at least one transducer capable of effectively applying a signal to neurons or a nerve. In those embodiments in which the neuromodulation apparatus is at least partially implanted in the subject, the elements of the apparatus that are to be implanted in the subject are constructed such that they are suitable for such implantation. Such suitable constructions would be well known to the skilled person. Indeed, various fully implantable neuromodulation apparatuses are currently available, such as the vagus nerve stimulator of SetPoint Medical, in clinical development for the treatment of rheumatoid arthritis (Arthritis & Rheumatism, Volume 64, No. 10 (Supplement), page S195 (Abstract No. 451), October 2012. “Pilot Study of Stimulation of the Cholinergic Anti-Inflammatory Pathway with an Implantable Vagus Nerve Stimulation Device in Patients with Rheumatoid Arthritis”, Frieda A. Koopman et al), and the INTERSTIM™ device (Medtronic, Inc), a fully implantable device utilised for sacral nerve modulation in the treatment of overactive bladder.

As used herein, “implanted” is taken to mean positioned at least partially within the subject's body. Partial implantation means that only part of the apparatus is implanted—i.e. only part of the device is positioned within the subject's body, with other elements of the apparatus external to the subject's body. Wholly implanted means that the entire of the apparatus is positioned within the subject's body. For the avoidance of doubt, the apparatus being “wholly implanted” does not preclude additional elements, independent of the apparatus but in practice useful for its functioning (for example, a remote wireless charging unit or a remote wireless manual override unit), being independently formed and external to the subject's body.

The present disclosure provides systems and methods for the treatment and/or prevention of xerostomia, particularly xerostomia associated with Sjögren's Syndrome, by stimulation of neural activity in the superior cervical ganglia (SCG). The postganglionic neurons of the SCG innervate the submandibular gland(s) via the cervical sympathetic trunk submandibular gland (CST-SMG) axis thereby regulating salivary production by these glands. Selective stimulation of postganglionic neurons innervating the salivary glands, e.g., the postganglionic neurons innervating the submandibular gland(s), is useful for the treatment of xerostomia, for example by increasing saliva secretion. Similarly, stimulation of SCG preganglionic neurons (CSC) can also be useful due to the same effects arising from downstream stimulation of those postganglionic neurons innervating the SMG(s).

It is particularly advantageous to stimulate neural activity in the SCG to treat xerostomia associated with Sjögren's Syndrome or to treat other symptoms of SS such as inflammation. The submandibular gland secretes a prohormone, submandibular rat-1 protein (SMR1), that is the precursor to several different bioactive peptides including sialorphin, submandibular gland peptide-T (SGP-T) and the tripeptide FEG. FEG and its metabolically stable D-isomeric peptide feG are potent inhibitors of inflammation. Human protein CABS1 is similar in structure to SMR1 and is thought to have similar anti-inflammatory effects. Therefore stimulation of the CST-SMG axis can be used to treat multiple aspects of Sjögren's Syndrome pathology including the dysregulated immune function characteristic of this disease. In particular, stimulation of the CST-SMG axis can treat SS by increasing saliva production to alleviate oral dryness and/or by increasing the secretion of local anti-inflammatory peptides to decrease inflammation.

A neuromodulation apparatus that stimulates neural activity in the superior cervical ganglia and/or in preganglionic and/or postganglionic neurons thereof will provide an effective treatment for xerostomia, particularly xerostomia associated with Sjögren's Syndrome. Such a neuromodulation apparatus also provides an effective treatment of SS by increasing saliva production and/or increasing anti-inflammatory peptide production in the submandibular gland(s) and/or saliva.

Such an apparatus can be advantageously used in conjunction with medications known for the treatment of xerostomia including but not limited to saliva substitutes, saliva stimulants and/or cholinergic parasympathomimetic agents such as Pilocarpine. Commercially available saliva substitutes include but are not limited to: Carboxymethyl or hydroxyethylcellulose solutions; Entertainer's Secret® (KLI Corp); Glandosane® (Kenwood/Bradley); Moi-Stir® (Kingswood Labs); Moi-Stir® Oral Swabsticks (Kingswood Labs); Optimoist® (Colgate-Palmolive); Saliva Substitute® (Roxane Labs); Salivart® (Gebauer) preservative-free aerosol; Salix® (Scandinavian Natural Health & Beauty) tablets; V. A. Oralube® (Oral Dis. Res. Lab) sodium-free liquid; Xero-Lube® Artificial Saliva (Scherer) sodium-free spray; Mucopolysaccharide Solutions; MouthKote® (Parnell) spray.

The apparatus may also be advantageously used in conjunction with medications known for the treatment of the systemic inflammation or conditions associated with Sjögren's Syndrome, for example immunosuppressants such as monoclonal antibodies (e.g. belimumab, rituximab) or drugs such as methotrexate.

Apparatus or system and methods in accordance with the invention can be used by subjects chronically using medications to alleviate xerostomia symptoms and/or other symptoms of Sjögren's Syndrome, for example inflammation. By using the apparatus or method of the invention, it is expected that the amount and/or frequency of administration of medications can be reduced, thereby improving subject compliance and minimising any negative side-effects associated with existing medications.

This disclosure teaches an apparatus or system for stimulating neural activity in a superior cervical ganglion (SCG) of a subject, the apparatus comprising one or more neural interfacing elements including transducers configured to apply a signal to one or more of the SCG, and/or preganglionic and/or postganglionic neurons thereof of the subject, a controller operably coupled to the one or more neural interfacing elements. The controller controls the signal to be applied by each of the one or more transducers, such that the signal stimulates the neural activity of the SCG to produce a physiological response in the subject. Such an apparatus or system is favourably used for the treatment of xerostomia, such as xerostomia associated with Sjögren's Syndrome. Such an apparatus may also be used to treat other symptoms of Sjögren's Syndrome, for example inflammation (e.g. oral inflammation, systemic inflammation).

In certain embodiments, the signal selectively stimulates neural activity in neurons innervating the salivary gland(s), preferably in neurons innervating the submandibular gland(s). In certain embodiments, the signal selectively stimulates neural activity in SCG, e.g., the preganglionic and/or postganglionic neurons forming part of the CST-SMG axis. In certain embodiments, the signal selectively stimulates neural activity in postganglionic neurons of the superior cervical ganglion wherein said postganglionic neurons are low-threshold secretomotor neurons.

In certain such embodiments, the signal applied by the one or more neural interfacing elements is an electrical signal, electromagnetic signal, an optical signal, a mechanical signal, an ultrasonic signal, or a thermal signal. In those embodiments in which the apparatus has at least two transducers, the signal which each of the transducers is configured to apply is independently selected from an electrical signal, an optical signal, an ultrasonic signal, and a thermal signal. That is, each transducer may be configured to apply a different signal. Alternatively, in certain embodiments each transducer is configured to apply the same signal.

In certain embodiments, each of the one or more transducers may be comprised of one or more electrodes, one or more photon sources, one or more ultrasound transducers, one more sources of heat, or one or more other types of transducer arranged to put the signal into effect.

In certain embodiments, the signal or signals applied by the one or more transducers is an electrical signal, for example a voltage or current. In such embodiments, the one or more transducers configured to apply the electrical signal are electrodes, for example wire electrodes or cuff electrodes. In certain such embodiments the signal applied comprises a direct current (DC) waveform, such as a charge balanced direct current waveform, or an alternating current (AC) waveform, or both a DC and an AC waveform.

In certain embodiments, the DC waveform or AC waveform may be a square, sinusoidal, triangular or complex waveform. The DC waveform may alternatively be a constant amplitude waveform. In certain embodiments the electrical signal is a DC square waveform of varying voltage.

In certain embodiments, the signal comprises an AC or DC waveform having a frequency in the range of 1 Hz-1 kHz, optionally 1-500 Hz, optionally 1-200 Hz, optionally 1-100 Hz, optionally 1-50 Hz, optionally 1-20 Hz, optionally 1-10 Hz, optionally 5-10 Hz, optionally 5 Hz or 7.5 Hz. In certain preferred embodiments the signal comprises a DC waveform having a frequency of 50-150 Hz. In certain preferred embodiments the signal comprises a DC waveform having a frequency of 100 Hz. It will be appreciated by those of skill in the art that the lower and upper limits of such ranges can vary independently, such that the signal can have a frequency of at least 1 Hz, or at least 5 Hz, or at least 25 Hz, or at least 50 Hz, or at least 100 Hz. Such a signal can have a frequency not to exceed 1 kHz, or 500 Hz, or 200 Hz, or 100 Hz, or 50 Hz or 20 Hz, or 10 Hz.

It will be appreciated by the skilled person that the current amplitude of an applied electrical signal necessary to achieve the intended stimulation will depend upon the positioning of the electrode and the associated electrophysiological characteristics (e.g. impedance). It is within the ability of the skilled person to determine the appropriate current amplitude for achieving the intended stimulation in a given subject. For example, the skilled person is aware of methods suitable to monitor the neural activity profile induced by neuronal or nerve stimulation.

In certain embodiments, the electrical signal comprises a DC waveform and/or an AC waveform having a current of 10-2000 μA, optionally 20-1000 μA, optionally 10-2000 μA, optionally 20-1000 μA, optionally 50-1000 μA, optionally 100-1000 μA, optionally 500-1000 μA, optionally 500-800 μA, optionally 800 μA, optionally 20-500 μA, optionally 50-250 μA. In certain embodiments the electrical signal has a current of at least 10 μA, at least 20 μA, at least 50 μA, at least 60 μA, at least 70 μA, at least 80 μA, at least 90 μA, at least 100 μA, at least 110 μA, at least 150 μA, at least 180 μA, at least 200 μA, at least 220 μA, at least 250 μA, at least 300 μA, at least 400 μA, at least 500 μA, at least 600 μA, at least 700 μA, at least 800 μA. These ranges are illustrative, and one of skill in the art will recognize that the lower and upper limits can vary independently.

In certain embodiments, the electrical signal comprises a DC waveform and/or an AC waveform having a pulse duration of duration of 0.001-5 ms, 0.01-5 ms, 0.1-5 ms, 1-5 ms, 1-2 ms, optionally 2 ms. in certain embodiments the pulse duration is 0.005-0.1 ms, optionally 0.01-0.05, optionally 0.01-0.04 ms, optionally 0.01-0.03 ms, optionally 0.01-0.02 ms, optionally 0.01 or 0.02 ms, or 0.04 ms.

In certain such embodiments, all the transducers are electrodes configured to apply an electrical signal, optionally the same electrical signal.

In certain embodiments wherein the signal applied by the one or more transducers is a thermal signal, the signal reduces the temperature of the neurons or nerve (i.e. cools the neurons or nerve).

In certain alternative embodiments, the signal increases the temperature of the neurons or nerve (i.e. heats the nerve). In certain embodiments, the signal both heats and cools the neurons or nerve.

In those embodiments in which the signal applied by the one or more transducers is a thermal signal, at least one of the one or more transducers is a transducer configured to apply a thermal signal. In certain such embodiments, all the transducers are configured to apply a thermal signal, optionally the same thermal signal.

In certain embodiments, one or more of the one or more transducers comprise a Peltier element configured to apply a thermal signal, optionally all of the one or more transducers comprise a Peltier element. In certain embodiments, one or more of the one or more transducers comprise a laser diode configured to apply a thermal signal, optionally all of the one or more transducers comprise a laser diode configured to apply a thermal signal. In certain embodiments, one or more of the one or more transducers comprise a electrically resistive element configured to apply a thermal signal, optionally all of the one or more transducers comprise a electrically resistive element configured to apply a thermal signal.

In certain embodiments the signal applied by the one or more transducers is a mechanical signal, optionally an ultrasonic signal. In certain alternative embodiments, the mechanical signal applied by the one or more transducers is a pressure signal.

In certain embodiments the signal applied by the one or more transducers is an electromagnetic signal, optionally an optical signal. In certain such embodiments, the one or more transducers comprise a laser and/or a light emitting diode configured to apply the optical signal.

In certain embodiments, the physiological response produced in the subject as a result of the stimulation caused by the signal is one or more of: treatment of xerostomia; treatment of Sjögren's Syndrome; an increase in saliva production; an increase in secretion from the salivary gland(s); an increase in secretion from the submandibular gland(s); an increase in total peptide production by the submandibular gland(s); an increase in anti-inflammatory peptide production by the submandibular gland(s); an increase in production of the pro-hormone submandibular rat-1 protein (SMR1)(or a human homolog thereof) and/or an increase in peptides derived therefrom including but not limited to sialorphin, SGP-T, FEG (or human homologs thereof); an increase in production of CABS1, opiorphin, and/or an increase in peptides derived therefrom; an alteration in the action potentials or pattern of action potentials in the postganglionic neurons of a superior cervical ganglion more closely resembling that exhibited by a healthy individual than before the application of the signal.

In certain embodiments, the apparatus further comprises a detector element to detect one or more physiological parameters in the subject. Such a detector element may be configured to detect the one or more physiological parameters. That is, in such embodiments each detector may detect more than one physiological parameter, for example two, three, four or all the detected physiological parameters. Alternatively, in such embodiments each of the one or more detector elements is configured to detect a separate parameter of the one or more physiological parameters detected.

In such certain embodiments, the controller is coupled to the detector element configured to detect one or more physiological parameters, and causes the transducer or transducers to apply the signal when the physiological parameter is detected to be meeting or exceeding a predefined threshold value.

In certain embodiments, the one or more detected physiological parameters are selected from: systemic sympathetic tone; salivary volume; total protein/peptide concentration of saliva; anti-inflammatory protein/peptide concentration of saliva; secretion from the salivary glands; secretion from the submandibular gland(s).

In certain embodiments, the one or more detected physiological parameters comprise an action potential or pattern of action potentials in neurons or a nerve of the subject, wherein the action potential or pattern of action potentials is associated with a subject having xerostomia, optionally a subject having xerostomia associated with Sjögren's Syndrome. In certain embodiments, the one or more detected physiological parameters comprise an action potential or pattern of action potentials in neurons or a nerve of the subject, wherein the action potential or pattern of action potentials is associated with a subject having Sjögren's Syndrome.

It will be appreciated that any two or more of the indicated physiological parameters may be detected in parallel or consecutively. For example, in certain embodiments, the controller is coupled to a detector or detectors configured to detect the pattern of action potentials the superior cervical ganglia (e.g. a postganglionic fiber thereof) and also the salivary production and/or secretion of the subject.

It has been identified that xerostomia and Sjögren's Syndrome can be relieved and/or prevented by stimulating neural activity in the sympathetic cervical ganglia—that is, by stimulating neural activity in neurons innervating the salivary gland(s), particularly neurons innervating the submandibular gland(s).

In certain embodiments, the signal is applied in response to a controller regulated by the subject (e.g., “on demand”). In such a case, it is advantageous that the subject is able to activate the signal in response to perception of xerostomia.

It is particularly advantageous to stimulate neural activity of postganglionic neurons innervating the submandibular gland(s) for treatment of xerostomia associated with Sjögren's Syndrome and/or for treatment of inflammation associated with Sjögren's Syndrome. In particular, secretions from the submandibular gland include peptides, specifically anti-inflammatory peptides, that may affect multiple aspects of disease pathology. It follows that stimulating the neural activity of neurons innervating the submandibular gland(s) may alleviate symptoms of Sjögren's Syndrome beyond xerostomia. Alternatively, or in addition, stimulating the neural activity of neurons innervating the submandibular gland(s) may alleviate the xerostomia associated with SS by multiple routes, for example by increasing saliva production/secretion and by increasing anti-inflammatory peptide production.

Stimulation of neural activity as a result of applying the signal is an increase in neural activity in the neurons or nerve to which the signal is applied. In certain embodiments, a signal may be applied to a nerve or nerves resulting in the neural activity in at least some of the neurons (for example, specific classes of neurons) being increased compared to the baseline neural activity in that part of the nerve. Stimulation of neural activity could equally be across the whole nerve, in which case neural activity would be increased for all neurons across the whole nerve or nerves.

In certain embodiments, the signal stimulates, preferably selectively stimulates, neural activity in postganglionic neurons of the SCG innervating the salivary gland(s). In certain preferred embodiments, the signal stimulates neural activity, preferably selectively stimulates neural activity, in postganglionic neurons innervating the submandibular gland i.e. postganglionic neurons forming the CST-SMG axis. In certain embodiments, the signal selectively stimulates neural activity in postganglionic neurons of the superior cervical ganglion wherein said postganglionic neurons are low-threshold secretomotor neurons.

In certain embodiments, the signal is applied to the specified neurons on the left-side of the subject, the specified neurons on the right-side of the subject, or both. That is, in certain embodiments the signal is applied unilaterally or, alternatively, bilaterally.

In certain embodiments, application of the signal to neurons, a nerve or nerves results in the modulation in neural activity that is an alteration to the pattern of action potentials in all or part of the neurons, nerve or nerves. In certain such embodiments, the neural activity is modulated such that the resultant pattern of action potentials in the neurons, nerve or nerves resembles the pattern of action potentials in the neurons, nerve or nerves observed in a healthy subject.

Modulation of neural activity may comprise altering the neural activity in various other ways, for example increasing or inhibiting a particular part of the activity and stimulating new elements of activity, for example in particular intervals of time, in particular frequency bands, according to particular patterns and so forth. Such altering of neural activity may for example represent both increases and/or decreases with respect to the baseline activity.

In certain embodiments, the controller causes the signal to be applied intermittently. In certain such embodiments, the controller causes the signal to applied for a first time period, then stopped for a second time period, then reapplied for a third time period, then stopped for a fourth time period. In such an embodiment, the first, second, third and fourth periods run sequentially and consecutively.

The series of first, second, third and fourth periods amounts to one application cycle. In certain such embodiments, multiple application cycles can run consecutively such that the signal is applied in phases, between which phases no signal is applied.

In certain embodiments, the application cycles are not immediately consecutive. In certain such embodiments the application cycles are separated by a period of 1-60 min, 5-30 min, 10-20 min, optionally 15 min.

In such embodiments, the duration of the first, second, third and fourth time periods is independently selected. That is, the duration of each time period may be the same or different to any of the other time periods. In certain such embodiments, the duration of each of the first, second, third and fourth time periods is any time from 5 seconds (5s) to 24 hours (24h), 30s to 12 h, 1 min to 12 h, 5 min to 8 h, 5 min to 6 h, 10 min to 6 h, 10 min to 4 h, 30 min to 4 h, 1 h to 4 h. In certain embodiments, the duration of each of the first, second, third and fourth time periods is 5s, 10s, 30s, 60s, 2 min, 5 min, 10 min, 20 min, 30 min, 40 min, 50 min, 60 min, 90 min, 2 h, 3 h, 4 h, 5 h, 6 h, 7 h, 8 h, 9 h, 10 h, 11h, 12 h, 13 h, 14 h, 15 h, 16 h, 17 h, 18 h, 19 h, 20 h, 21 h, 22 h, 23 h, 24 h.

In certain embodiments, consecutive application cycles are applied for an operative period—that is, an operative period is a period over which consecutive application cycles are in operation. In such embodiments, the operative period is immediately followed by an inoperative period. In certain embodiments, the operative and inoperative period have a duration independently selected from 1-60 min, 5-30 min, 10-20 min, optionally 15 min. In certain embodiments, the operative and inoperative period have a duration independently selected from 1-24h, 1-12h, 1-6h, optionally 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21h, 22 h, 23 h, 24 h.

In certain embodiments wherein the controller causes the signal to be applied intermittently, the signal is applied for a specific amount of time per day. In certain such embodiments, the signal is applied for 10 min, 20 min, 30 min, 40 min, 50 min, 60 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h per day. In certain such embodiments, the signal is applied continuously for the specified amount of time. In certain alternative such embodiments, the signal may be applied discontinuously across the day, provided the total time of application amounts to the specified time.

In certain embodiments wherein the controller causes the signal to be applied intermittently, the signal is applied only when the subject is in a specific physiological state. In certain such embodiments, the signal is applied only when the subject exhibits xerostomia or is identified as having Sjögren's Syndrome.

In certain such embodiments, the apparatus further comprises a communication, or input, element via which the status of the subject (e.g. that they are experiencing xerostomia) can be indicated by the subject or a physician. In alternative embodiments, the apparatus further comprises a detector configured to detect the status of the subject, wherein the signal is applied only when the detector detects that the subject is in the specific state.

In certain alternative embodiments, the controller causes the signal to be permanently applied. That is, once begun, the signal is continuously applied to the neurons, nerve or nerves. It will be appreciated that in embodiments wherein the signal is a series of pulses, gaps between pulses do not mean the signal is not continuously applied.

In certain embodiments of the apparatus, the modulation in neural activity caused by the application of the signal is temporary. That is, upon cessation of the signal, neural activity in the neurons, nerve or nerves returns substantially towards baseline neural activity within 1-60 seconds, or within 1-60 minutes, or within 1-24 hours, optionally 1-12 hours, optionally 1-6 hours, optionally 1-4 hours, optionally 1-2 hours. In certain such embodiments, the neural activity returns substantially fully to baseline neural activity. That is, the neural activity following cessation of the signal is substantially the same as the neural activity prior to the signal being applied—i.e. prior to modulation. For example, the signal can be applied for a predetermined time period in response to a manipulation by the subject that indicates to the controller to apply the signal.

In certain alternative embodiments, the modulation in neural activity caused by the application of the signal or signals is substantially persistent. That is, upon cessation of the signal, neural activity in the neurons, nerve or nerves remains substantially the same as when the signal was being applied—i.e. the neural activity during and following modulation is substantially the same.

In certain embodiments, the modulation in neural activity caused by the application of the signal is partially corrective, preferably substantially corrective. That is, upon cessation of the signal, neural activity in the neurons, nerve or nerves more closely resembles the pattern of action potentials in the nerve(s) observed in a healthy subject than prior to modulation, preferably substantially fully resembles the pattern of action potentials in the nerve(s) observed in a healthy subject. In such embodiments, the modulation caused by the signal can be any modulation as defined herein. For example, application of the signal may result in stimulation of neural activity, and upon cessation of the signal, the pattern of action potentials in the neurons, nerve or nerves resembles the pattern of action potentials observed in a healthy individual. It is hypothesised that such a corrective effect is the result of a positive feedback loop—that is, the underlying cause of or predisposition to xerostomia, for example as a result of Sjögren's Syndrome, is treated as result of the apparatus and the claimed methods.

In certain embodiments, the apparatus is suitable for at least partial implantation into the subject. In certain such embodiments, the apparatus is suitable to be wholly implanted in the subject.

In certain embodiments, the apparatus further comprises one or more power supply elements, for example a battery, and/or one or more communication elements.

In another aspect, the invention provides a method for treating xerostomia in a subject, for example xerostomia associated with Sjögren's Syndrome, the method comprising implanting an apparatus as described above, positioning at least one transducer of the apparatus in contact with neurons of a superior cervical ganglion (SCG) of a subject, and activating the apparatus. In a further aspect, the invention provides a method for treating Sjögren's Syndrome in a subject, the method comprising implanting an apparatus as described above, positioning at least one transducer of the apparatus in contact with neurons of a superior cervical ganglion (SCG) of a subject, and activating the apparatus. In such aspects, the transducer is in signalling contact with the neurons when it is positioned such that the signal can be effectively applied to the neurons. The apparatus is activated when the apparatus is in an operating state such that the signal will be applied as determined by the controller, optionally in response to a manipulation by the subject.

In certain such embodiments, a first transducer is positioned in signalling contact with neurons of the left superior cervical ganglion of said subject to stimulate neural activity in said neurons of said left superior cervical ganglion in the subject, and a second transducer is positioned in signalling contact with neurons of the right superior cervical ganglion in said subject to stimulate neural activity in said neurons of said right superior cervical ganglion in the subject. In certain such embodiments, the first and second transducers are part of one apparatus or system according to the description above. In alternative embodiments, the first and second transducers are part of separate apparatuses or systems.

In certain embodiments, the neurons are postganglionic neurons of the SCG that innervate the salivary gland(s), preferably the submandibular gland(s). In certain embodiments, the postganglionic neurons form part of the CST-SMG neuroendocrine axis.

In certain embodiments, the method further comprises administration of a saliva substitute, a saliva stimulant, a cholinergic parasympathomimetic agent, and/or an immunosuppressant as described elsewhere herein.

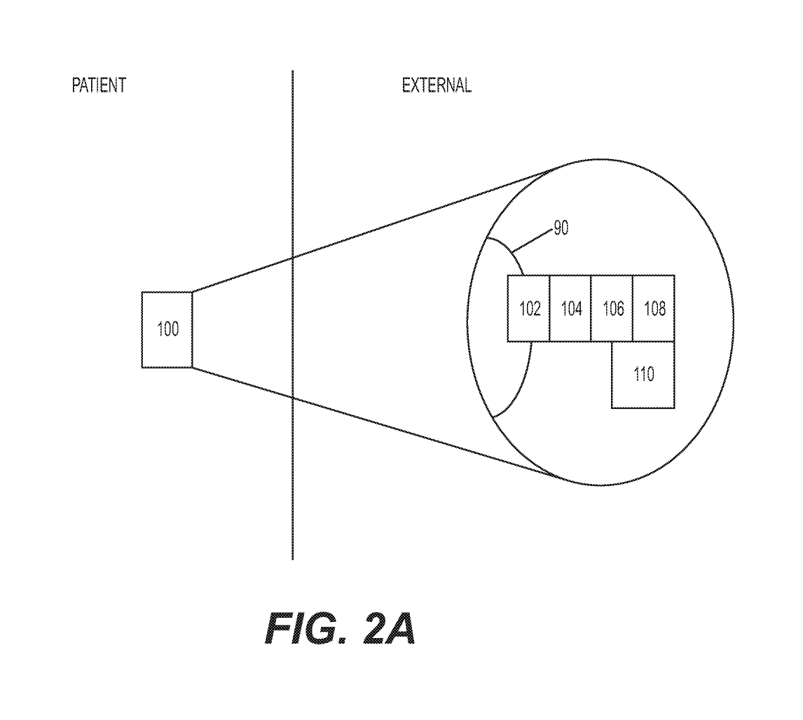

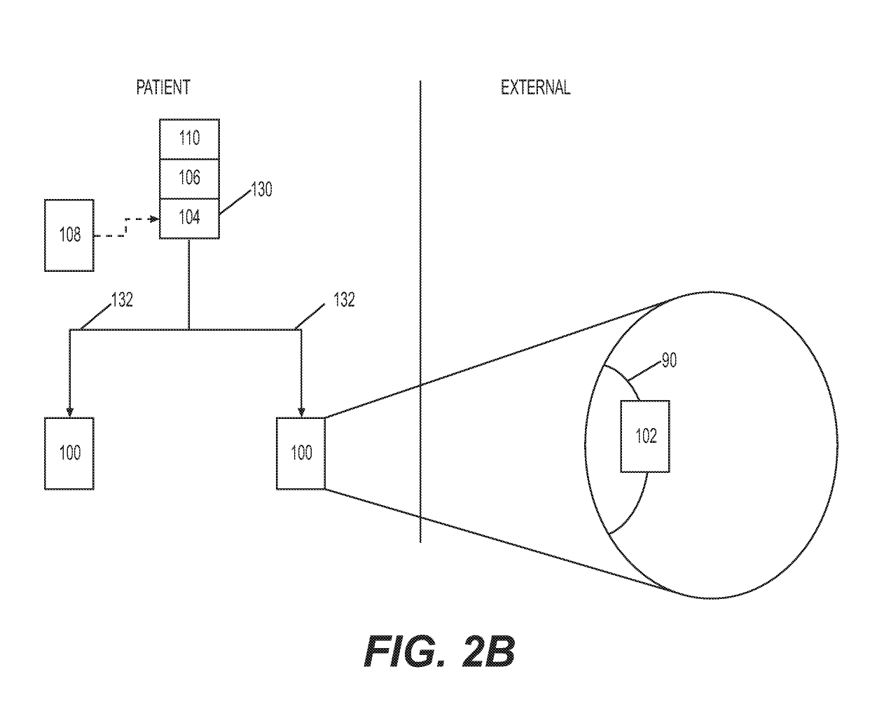

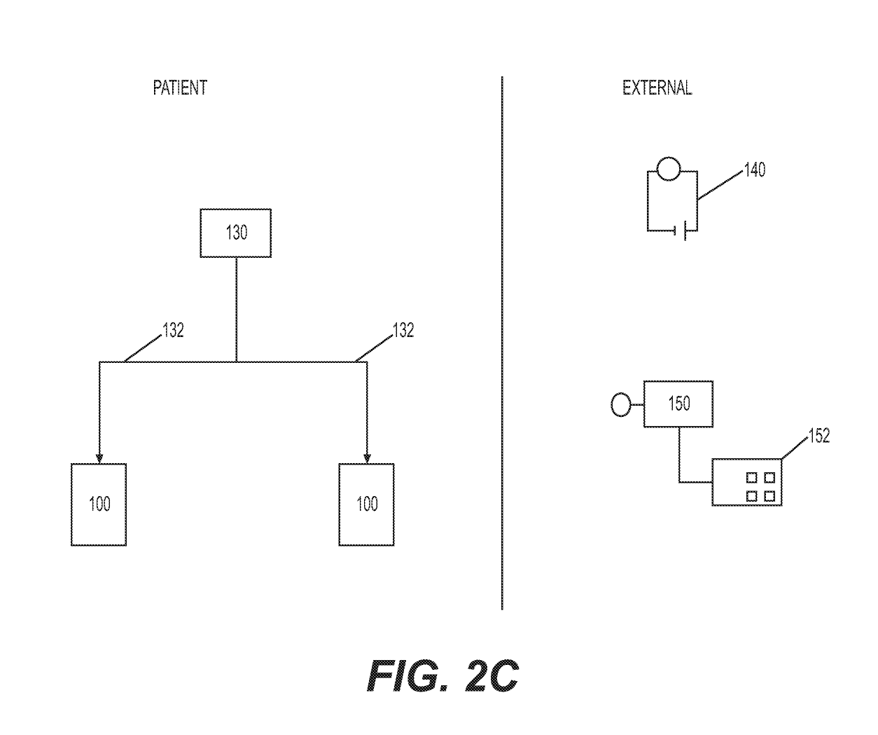

Implementation of all aspects of the invention (as discussed both above and below) will be further appreciated by reference to FIGS. 2A-2C.

FIGS. 2A-2C show how the invention may be put into effect using one or more neuromodulation apparatuses which are implanted in, located on, or otherwise disposed with respect to a subject in order to carry out any of the various methods described herein. In this way, one or more neuromodulation apparatuses/devices can be used to treat Sjögren's Syndrome or xerostomia in a subject, for example xerostomia associated with Sjögren's Syndrome, by stimulating neural activity in neurons of a superior cervical ganglion, for example preganglionic neurons or postganglionic neurons innervating the submandibular gland i.e. neurons forming part of the CST-SMG axis.

In each of the FIGS. 2B-2C a separate neuromodulation apparatus 100 is provided in respect of each of the left and right superior cervical ganglia, although as discussed herein an apparatus could be provided or used in respect of only one of the left and right superior cervical ganglia. Each such neuromodulation apparatus may be fully or partially implanted in the subject, or otherwise located, so as to provide neuromodulation of the respective neurons. Each of the left and right neuromodulation apparatuses 100 may operate independently, or may operate in communication with each other.

FIG. 2A also shows schematically components of an implanted neuromodulation apparatus 100, in which the apparatus comprises several elements, components or functions grouped together in a single unit and implanted in the subject. A first such element is a transducer 102 which is shown in proximity to postganglionic neurons of a superior cervical ganglion of the subject 90. The transducer 102 may be operated by a controller element 104. The apparatus may comprise one or more further elements such as a communication element 106, a detector element 108, a power supply element 110 and so forth.

Each neuromodulation apparatus 100 may carry out the required neuromodulation (i.e. stimulation) independently, or in response to one or more control signals. Such a control signal may be provided by the controller element 104 according to an algorithm, in response to output of one or more detector elements 108, and/or in response to communications from one or more external sources received using the communications element. As discussed herein, the detector element(s) could be responsive to a variety of different physiological parameters.

FIG. 2B illustrates some ways in which the apparatus of FIG. 2A may be differently distributed. For example, in FIG. 2B the neuromodulation apparatuses 100 comprise transducers 102 implanted proximally to postganglionic neurons of a superior cervical ganglion 90, but other elements such as a controller element 104, a communication element 106 and a power supply element 110 are implemented in a separate control unit 130 which may also be implanted in, or carried by the subject. The separate control unit 130 then controls the transducers in both of the neuromodulation apparatuses via connections 132 which may for example comprise electrical wires and/or optical fibres for delivering signals and/or power to the transducers.

In the arrangement of FIG. 2B one or more detector elements 108 are located separately from the separate control unit 130, although one or more such detector elements could also or instead be located within the separate control unit 130 and/or in one or both of the neuromodulation apparatuses 100. The detector elements may be used to detect one or more physiological parameters of the subject, and the controller element or control unit then causes the transducers to apply the signal in response to the detected parameter(s), for example only when a detected physiological parameter meets or exceeds a predefined threshold value. Physiological parameters which could be detected for such purposes include systemic sympathetic tone; salivary volume; total protein/peptide concentration of saliva; anti-inflammatory protein/peptide concentration of saliva; secretion from the salivary gland(s); secretion from the submandibular gland(s).

A variety of other ways in which the various functional elements could be located and grouped into the neuromodulation apparatuses, a separate control unit 130 and elsewhere are of course possible. For example, one or more sensors of FIG. 2B could be used in the arrangement of FIG. 2A or 2C or other arrangements.

FIG. 2C illustrates some ways in which some functionality of the apparatus of FIG. 2A or 2B is provided not implanted in the subject. For example, in FIG. 2C an external power supply 140 is provided which can provide power to implanted elements of the apparatus in ways familiar to the skilled person, and an external controller 150 provides part or all of the functionality of the controller element 104, and/or provides other aspects of control of the apparatus, and/or provides data readout from the apparatus, and/or provides a data input facility 152. The data input facility could be used by a subject or other operator in various ways, for example to input data relating to the status of the subject (e.g. if they are experiencing xerostomia).

Each neuromodulation apparatus may be adapted to carry out the neuromodulation required (i.e. stimulation, for example selective stimulation) using one or more physical modes of operation which typically involve applying a signal to postganglionic neurons of a superior cervical ganglion, such a signal typically involving a transfer of energy to (or from) the neurons. As already discussed, such modes may comprise stimulating the neurons, nerve or nerves using an electrical signal, an optical signal, an ultrasound or other mechanical signal, a thermal signal, a magnetic or electromagnetic signal, or some other use of energy to carry out the required modulation. Such signals may be non-destructive signals. To this end, the transducer 102 illustrated in FIG. 2A could be comprised of one or more electrodes, one or more photon sources, one or more ultrasound transducers, one more sources of heat, or one or more other types of transducer arranged to put the required neuromodulation (i.e. stimulation of neural activity) into effect. Preferably the apparatus is comprised of one or more electrodes configured to apply an electrical signal, for example a wire electrode or a cuff electrode.

The neural modulation device(s) or apparatus may be arranged to stimulate neural activity in neurons of a superior cervical ganglion by using the transducer(s) to apply a voltage or current, for example a direct current (DC) waveform, such as a charge balanced direct current, or an AC waveform, or both.

In certain embodiments, the DC waveform or AC waveform may be a square, sinusoidal, triangular or complex waveform. The DC waveform may alternatively be a constant amplitude waveform. In certain embodiments the electrical signal is a DC square waveform of varying voltage.

In certain embodiments, the signal comprises an AC or DC waveform having a frequency in the range of 1 Hz-1 kHz, optionally 1-500 Hz, optionally 1-200 Hz, 1-100 Hz, 1-50 Hz, 1-20 Hz, 1-10 Hz, 5-10 Hz, optionally 5 Hz or 7.5 Hz. In certain preferred embodiments the signal comprises a DC waveform having a frequency of 50-150 Hz. In certain preferred embodiments the signal comprises a DC waveform having a frequency of 100 Hz. It will be appreciated by those of skill in the art that the lower and upper limits of such ranges can vary independently, such that the signal can have a frequency of at least 1 Hz, or at least 5 Hz, or at least 25 Hz, or at least 50 Hz, or at least 100 Hz. Such a signal can have a frequency not to exceed 1 kHz, or 500 Hz, or 200 Hz, or 100 Hz, or 50 Hz, or 10 Hz, or 8 Hz.

It will be appreciated by the skilled person that the current amplitude of an applied electrical signal necessary to achieve the intended stimulation will depend upon the positioning of the electrode and the associated electrophysiological characteristics (e.g. impedance). It is within the ability of the skilled person to determine the appropriate current amplitude for achieving the intended stimulation in a given subject. For example, the skilled person is aware of methods suitable to monitor the neural activity profile induced by neuronal or nerve stimulation.

In certain embodiments, the electrical signal comprises a DC waveform and/or an AC waveform having a current of 10-2000 μA, optionally 20-1000 μA, 50-1000 μA, 100-1000 μA, 500-1000 μA, 500-800 μA, optionally 800 μA. In certain embodiments the electrical signal has a current of 20-500 μA, optionally 50-250 μA. In certain embodiments the electrical signal has a current of at least 10 μA, at least 20 μA, at least 50 μA, at least 60 μA, at least 70 μA, at least 80 μA, at least 90 μA, at least 100 μA, at least 110 μA, at least 150 μA, at least 180 μA, at least 200 μA, at least 220 μA at least 250 μA, at least 300 μA, at least 400 μA, at least 500 μA, at least 600 μA, at least 700 μA, at least 800 μA. These ranges are illustrative, and one of skill in the art will recognize that the lower and upper limits can vary independently.

In certain embodiments, the electrical signal comprises a DC waveform and/or an AC waveform having a pulse duration of duration of 0.001-5 ms, 0.01-5 ms, 0.1-5 ms, 1-5 ms, 1-2 ms, optionally 2 ms.

In certain embodiments the pulse duration is 0.005-0.1 ms, optionally 0.01-0.05, optionally 0.01-0.04 ms, optionally 0.01-0.03 ms, optionally 0.01-0.02 ms, optionally 0.01 or 0.02 ms, or 0.04 ms.

Optogenetics is a technique that genetically modifies cells to express photosensitive features, which can then be activated with light to modulate cell function. Many different optogenetic tools have been developed that can be used to modulate neural firing. Mechanical forms of neuromodulation can include the use of ultrasound which may conveniently be implemented using external instead of implanted ultrasound transducers. Other forms of mechanical neuromodulation include the use of pressure (for example see “The effects of compression upon conduction in myelinated axons of the isolated frog sciatic nerve” by Robert Fern and P. J. Harrison Br.j. Anaesth. (1975), 47, 1123, which is incorporated herein by reference).

The techniques discussed above principally relate to the stimulation of neuronal activity. Where modulation by inhibition or blocking of neural activity or otherwise modifying activity in various ways is required, electrodes adjacent to or in contact with the neurons, nerve or particular parts of the nerve may be used to impart an electrical signal to inhibit activity in various ways, as would be appreciated by the skilled person.

In another aspect, the invention provides a method of treating xerostomia in a subject, for example xerostomia associated with Sjögren's Syndrome, the method comprising applying a signal to neurons of a superior cervical ganglion of said subject to stimulate neural activity in said neurons in the subject. In certain embodiments, the signal is applied to preganglionic neurons and/or postganglionic neurons innervating the salivary gland(s), preferably the submandibular gland(s).

In a further aspect, the invention provides a method of treating Sjögren's Syndrome in a subject, the method comprising applying a signal to neurons of a superior cervical ganglion of said subject to stimulate neural activity in said neurons in the subject. In certain embodiments, the signal is applied to preganglionic and/or postganglionic neurons innervating the salivary gland(s), preferably the submandibular gland(s).

In certain embodiments, the signal is applied by a neuromodulation apparatus comprising one or more transducers configured to apply the signal. In certain preferred embodiments the neuromodulation apparatus is at least partially implanted in the subject. In certain preferred embodiments, the neuromodulation apparatus is wholly implanted in the subject.

In certain embodiments, the treatment of xerostomia is prophylactic treatment. That is, the methods of the invention reduce the likelihood of a subject developing xerostomia, for example if said subject is prescribed a medication known to have xerostomia as a side-effect. The methods of the invention may be used to prevent xerostomia associated with Sjögren's Syndrome in subjects identified as at higher risk of developing this disease as compared with the average population risk.

In certain embodiments, the treatment of xerostomia is therapeutic treatment. That is, the methods of the invention at least partially relieve or ameliorate the severity of xerostomia in subjects exhibiting oral dryness, for example as a side-effect of treatment or as a symptom of another disease, particularly Sjögren's Syndrome. In methods of the invention used to treat xerostomia associated with SS, the alleviation of oral dryness may be accompanied by a decrease in inflammation, for example oral inflammation and/or systemic inflammation.

In certain embodiments, treatment of xerostomia, for example xerostomia associated with Sjögren's Syndrome, is indicated by an improvement in a measurable physiological parameter, for example a decrease in systemic sympathetic tone; an increase in salivary volume; an increase in the protein/peptide concentration of saliva (for example an increase in total protein concentration and/or an increase in an anti-inflammatory protein or peptide (e.g. SMR1, CABS1, FEG, feG, sialorphin, opiorphin or homologs thereof)); an increase in secretion from the salivary glands; and/or an increase in secretion from the submandibular gland(s). In SS, an improvement in a measurable parameter may be a decrease in inflammation, for example oral inflammation and/or systemic inflammation, optionally accompanied by an improvement in one or more of the parameters recited above.

In certain embodiments, the treatment of Sjögren's Syndrome is prophylactic treatment. That is, the methods of the invention reduce the likelihood of a Sjögren's Syndrome patient developing one or more symptoms of Sjögren's Syndrome, for example xerostomia, ocular dryness, or oral inflammation.

In certain embodiments, the treatment of Sjögren's Syndrome is therapeutic. That is, the methods of the invention at least partially relieve or ameliorate one or more symptoms of Sjögren's Syndrome, for example oral dryness, oral inflammation, systemic inflammation.

In certain embodiments, treatment of xerostomia, for example xerostomia associated with Sjögren's Syndrome, is indicated by an improvement in a measurable physiological parameter, for example a decrease in systemic sympathetic tone; an increase in salivary volume; an increase in the protein/peptide concentration of saliva (for example an increase in total protein concentration and/or an increase in an anti-inflammatory protein or peptide (e.g. SMR1, CABS1, FEG, feG, sialorphin, opiorphin or homologs thereof)); an increase in secretion from the salivary glands; and/or an increase in secretion from the submandibular gland(s). In SS, an improvement in a measurable parameter may be a decrease in inflammation, for example oral inflammation and/or systemic inflammation, optionally accompanied by an improvement in one or more of the parameters recited above.

Suitable methods for determining the value for any given parameter would be appreciated by the skilled person and have been described above.

In certain embodiments, treatment of the condition is indicated by an improvement in the profile of neural activity in the neurons, nerve or nerves to which the signal is applied. That is, treatment of the condition is indicated by the neural activity in the neurons or nerve(s) approaching the neural activity in a healthy individual—i.e. the pattern of action potentials in the nerve more closely resembling that exhibited by a healthy individual than before the intervention.

Stimulation of neural activity as a result of applying the signal is an increase in neural activity in the neurons or nerve to which the signal is applied. In certain embodiments, a signal may be applied to a nerve or nerves resulting in the neural activity in at least some of the neurons (for example, specific classes of neurons) being increased compared to the baseline neural activity in that part of the nerve. Stimulation of neural activity could equally be across the whole nerve, in which case neural activity would be increased for all neurons across the whole nerve or nerves.

In certain embodiments, the signal stimulates, preferably selectively stimulates, neural activity in postganglionic neurons of the SCG innervating the salivary gland(s). In certain preferred embodiments, the signal stimulates neural activity, preferably selectively stimulates neural activity, in postganglionic neurons innervating the submandibular gland i.e. postganglionic neurons forming the CST-SMG axis. In certain embodiments, the signal selectively stimulates neural activity in postganglionic neurons of the superior cervical ganglion wherein said postganglionic neurons are low-threshold secretomotor neurons.

In certain embodiments, the signal is applied to the specified neurons or a nerve on the left-side of the subject, the specified neurons or a nerve on the right-side of the subject, or both. That is, in certain embodiments the signal is applied unilaterally or, alternatively, bilaterally.

In certain embodiments, the signal is applied intermittently. In certain such embodiments, the signal is applied for a first time period, then stopped for a second time period, then reapplied for a third time period, then stopped for a fourth time period. In such an embodiment, the first, second, third and fourth periods run sequentially and consecutively. The series of first, second, third and fourth periods amounts to one application cycle. In certain such embodiments, multiple application cycles can run consecutively such that the signal is applied in phases, between which phases no signal is applied.

In certain embodiments, the application cycles are not immediately consecutive. In certain such embodiments the application cycles are separated by a period of 1-60 min, 5-30 min, 10-20 min, optionally 15 min.

In such embodiments, the duration of the first, second, third and fourth time periods is independently selected. That is, the duration of each time period may be the same or different to any of the other time periods. In certain such embodiments, the duration of each of the first, second, third and fourth time periods is any time from 5 seconds (5s) to 24 hours (24h), 30s to 12 h, 1 min to 12 h, 5 min to 8 h, 5 min to 6 h, 10 min to 6 h, 10 min to 4 h, 30 min to 4 h, 1 h to 4 h. In certain embodiments, the duration of each of the first, second, third and fourth time periods is 5s, 10s, 30s, 60s, 2 min, 5 min, 10 min, 20 min, 30 min, 40 min, 50 min, 60 min, 90 min, 2 h, 3 h, 4 h, 5 h, 6 h, 7 h, 8 h, 9 h, 10 h, 11h, 12 h, 13 h, 14 h, 15 h, 16 h, 17 h, 18 h, 19 h, 20 h, 21 h, 22 h, 23 h, 24 h.

In certain embodiments, consecutive application cycles are applied for an operative period—that is, an operative period is a period over which consecutive application cycles are in operation. In such embodiments, the operative period is immediately followed by an inoperative period. In certain embodiments, the operative and inoperative period have a duration independently selected from 1-60 min, 5-30 min, 10-20 min, optionally 15 min. In certain embodiments, the operative and inoperative period have a duration independently selected from 1-24h, 1-12h, 1-6h, optionally 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21h, 22 h, 23 h, 24 h.

In certain embodiments wherein the signal is applied intermittently, the signal is applied for a specific amount of time per day. In certain such embodiments, the signal is applied for 10 min, 20 min, 30 min, 40 min, 50 min, 60 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h per day. In certain such embodiments, the signal is applied continuously for the specified amount of time. In certain alternative such embodiments, the signal may be applied discontinuously across the day, provided the total time of application amounts to the specified time.

In certain embodiments wherein the signal is applied intermittently, the signal is applied only when the subject is in a specific state. In certain such embodiments, the signal is applied only when the subject exhibits xerostomia. In such embodiments, the status of the subject (e.g. that they are experiencing xerostomia) can be indicated by the subject. In such a case, the subject can control the controller to apply the signal to the SCG. In alternative such embodiments, the status of the subject can be detected independently from any input from the subject. In certain embodiments in which the signal is applied by a neuromodulation apparatus, the apparatus further comprises a detector configured to detect the status of the subject, wherein the signal is applied only when the detector detects that the subject is in the specific state.

In certain embodiments of methods according to the invention, the method further comprises the step of detecting one or more physiological parameters of the subject, wherein the signal is applied only when the detected physiological parameter meets or exceeds a predefined threshold value. In such embodiments wherein more than one physiological parameter is detected, the signal may be applied when any one of the detected parameters meets or exceeds its threshold value, alternatively only when all of the detected parameters meet or exceed their threshold values. In certain embodiments wherein the signal is applied by a neuromodulation apparatus, the apparatus further comprises at least one detector element configured to detect the one or more physiological parameters.

In certain embodiments, the one or more detected physiological parameters are selected from: systemic sympathetic tone; salivary volume; total protein/peptide concentration of saliva; anti-inflammatory protein/peptide concentration of saliva; secretion from the salivary gland(s); secretion from the submandibular gland(s).

Similarly, in certain embodiments the detected physiological parameter could be an action potential or pattern of action potentials in neurons or a nerve of the subject, for example postganglionic neurons of a superior cervical ganglion, wherein the action potential or pattern of action potentials is associated with xerostomia and/or Sjögren's Syndrome.

It will be appreciated that any two or more of the indicated physiological parameters may be detected in parallel or consecutively. For example, in certain embodiments, the pattern of action potentials in the postganglionic neurons of a superior cervical ganglion can be detected at the same time as secretion by the salivary gland(s), preferably the submandibular gland(s).

In certain embodiments, the subject activates the signal via the controller, e.g., in response to perception of a physiological parameter.

In certain embodiments, the signal is permanently applied. That is, once begun, the signal is continuously applied to the neurons, nerve or nerves. It will be appreciated that in embodiments wherein the signal is a series of pulses, gaps between pulses do not mean the signal is not continuously applied.

In certain embodiments of the methods, the stimulation in neural activity caused by the application of the signal is temporary. That is, upon cessation of the signal, neural activity in the nerve or nerves returns substantially towards baseline neural activity within 1-60 seconds, or within 1-60 minutes, or within 1-24 hours, optionally 1-12 hours, optionally 1-6 hours, optionally 1-4 hours, optionally 1-2 hours. In certain such embodiments, the neural activity returns substantially fully to baseline neural activity. That is, the neural activity following cessation of the signal is substantially the same as the neural activity prior to the signal being applied—i.e. prior to modulation.

In certain alternative embodiments, the stimulation of neural activity caused by the application of the signal is substantially persistent. That is, upon cessation of the signal, neural activity in the neurons, nerve or nerves remains substantially the same as when the signal was being applied—i.e. the neural activity during and following stimulation is substantially the same.

In certain embodiments, the stimulation of neural activity caused by the application of the signal is partially corrective, preferably substantially corrective. That is, upon cessation of the signal, neural activity in the neurons, nerve or nerves more closely resembles the pattern of action potentials observed in a healthy subject than prior to stimulation, preferably substantially fully resembles the pattern of action potentials observed in a healthy subject. For example, application of the signal stimulates neural activity, and upon cessation of the signal, the pattern of action potentials in the neurons, nerve or nerves resembles the pattern of action potentials observed in a healthy subject. It is hypothesised that such a corrective effect is the result of a positive feedback loop.

In certain such embodiments, once first applied, the signal may be applied intermittently or permanently, as described in the embodiments above.

In certain embodiments, the signal is applied to postganglionic neurons of one of the superior cervical ganglia. In certain embodiments, the signal selectively stimulates postganglionic neurons of the CST-SMG axis. In certain embodiments, the signal selectively stimulates neural activity in postganglionic neurons of the superior cervical ganglion wherein said postganglionic neurons are low-threshold secretomotor neurons.

In certain embodiments, the signal is applied bilaterally. That is, in such embodiments, the signal is applied to neurons on both the left and right side of the subject such that neural activity is stimulated in the neurons to which the signal is applied—i.e. the stimulation is bilateral. In such embodiments, the signal applied to right and left SCG, and therefore the extent of stimulation, is independently selected. In certain embodiments the signal applied to the neurons on the right side is the same as the signal applied to the neurons on the left side. In certain alternative embodiments the signal applied to the neurons on the right side is different to the signal applied to the neurons on the left side.

In certain embodiments wherein the modulation is bilateral, each signal is applied by a neuromodulation apparatus comprising one or more transducers for applying the signal. In certain such embodiments, all signals are applied by the same neuromodulation apparatus, that apparatus have at least two transducers, one to apply the signal to the left-side neurons and one to apply the signal to the right-side neurons. In certain alternative embodiments, each signal is applied by a separate neuromodulation apparatus.

In certain embodiments, the signal applied is a non-destructive signal.

In certain embodiments of the methods according to the invention, the signal applied is an electrical signal, an electromagnetic signal (optionally an optical signal), a mechanical (optionally ultrasonic) signal, a thermal signal, a magnetic signal or any other type of signal.

In certain such embodiments in which more than one signal may be applied, for example when the modulation is bilateral, each signal may be independently selected from an electrical signal, an optical signal, an ultrasonic signal, and a thermal signal. In those such embodiments in which two signals are applied by one modulation apparatus, the two signals may be the same type of signal or may be different types of signal independently selected from an electrical signal, an optical signal, an ultrasonic signal, and a thermal signal. In those embodiments in which two signals are applied, each by a separate neuromodulation apparatus, the two signals may be the same type of signal or may be different types of signal independently selected from an electrical signal, an optical signal, an ultrasonic signal, and a thermal signal.

In certain embodiments in which the signal is applied by a neuromodulation apparatus comprising at least one transducer, the transducer may be comprised of one or more electrodes, one or more photon sources, one or more ultrasound transducers, one more sources of heat, or one or more other types of transducer arranged to put the signal into effect.

In certain embodiments, the signal is an electrical signal, for example a voltage or current, and the transducer is an electrode, for example a wire electrode or a cuff electrode. In certain such embodiments the signal comprises a direct current (DC) waveform, such as a charge balanced DC waveform, or an alternating current (AC) waveform, or both a DC and an AC waveform.

In certain embodiments, the DC waveform or AC waveform may be a square, sinusoidal, triangular or complex waveform. The DC waveform may alternatively be a constant amplitude waveform. In certain embodiments the electrical signal is a DC square waveform of varying voltage.

In certain embodiments, the electrical signal comprises an AC waveform or a DC waveform having a frequency in the range of 1 Hz-1 kHz, optionally 1-500 Hz, optionally 1-200 Hz, 1-100 Hz, 1-50 Hz, 1-20 Hz, 1-10 Hz, 5-10 Hz, optionally 5 Hz or 7.5 Hz, optionally 50-150 Hz, optionally 100 Hz, or any alternative frequency within the lower and upper limits described.

It will be appreciated by the skilled person that the current amplitude of an applied electrical signal necessary to achieve the intended stimulation will depend upon the positioning of the electrode and the associated electrophysiological characteristics (e.g. impedance). It is within the ability of the skilled person to determine the appropriate current amplitude for achieving the intended stimulation in a given subject. For example, the skilled person is aware of methods suitable to monitor the neural activity profile induced by neuronal or nerve stimulation.

In certain embodiments, the electrical signal comprises a DC waveform and/or an AC waveform having a current of 10-2000 μA, optionally 20-1000 μA, 50-1000 μA, 100-1000 μA, 500-1000 μA, 500-800 μA, optionally 800 μA. In certain embodiments, the electrical signal has a current of 20-500 μA, optionally 50-250 μA. In certain embodiments the electrical signal has a current of at least 10 μA, at least 20 μA, at least 50 μA, at least 60 μA, at least 70 μA, at least 80 μA, at least 90 μA, at least 100 μA, at least 110 μA, at least 150 μA, at least 180 μA, at least 200 μA, at least 220 μA, at least 250 μA, at least 300 μA, at least 400 μA, at least 500 μA, at least 600 μA, at least 700 μA, at least 800 μA. These ranges are illustrative, and one of skill in the art will recognize that the lower and upper limits can vary independently.

In certain embodiments, the electrical signal comprises a DC waveform and/or an AC waveform having a pulse duration of duration of 0.001-5 ms, 0.01-5 ms, 0.1-5 ms, 1-5 ms, 1-2 ms, optionally 2 ms. in certain embodiments the pulse duration is 0.005-0.1 ms, optionally 0.01-0.05, optionally 0.01-0.04 ms, optionally 0.01-0.03 ms, optionally 0.01-0.02 ms, optionally 0.01 or 0.02 ms, or 0.04 ms.

In certain embodiments wherein the signal is a thermal signal, the signal reduces the temperature of the neurons or nerve (i.e. cools the neurons or nerve). In certain alternative embodiments, the signal increases the temperature of the neurons or nerve (i.e. heats the neurons or nerve). In certain embodiments, the signal both heats and cools the neurons or nerve.

In certain embodiments wherein the signal is a mechanical signal, the signal is an ultrasonic signal. In certain alternative embodiments, the mechanical signal is a pressure signal.

In certain embodiments, the method further comprises administration of a saliva substitute or saliva stimulant to the subject.

In another aspect, the invention provides a saliva substitute or saliva stimulant for use in a method of treating xerostomia in a subject, wherein the method comprises:
i. applying a signal to a superior cervical ganglion of said subject to stimulate neural activity in neurons of the SCG; and
ii. administering the saliva substitute or saliva stimulant to the subject.

In another aspect, the invention provides a saliva substitute or saliva stimulant for use in a method of treating Sjögren's Syndrome in a subject, wherein the method comprises:
i. applying a signal to a superior cervical ganglion (SCG) of said subject to stimulate neural activity in said SCG; and
ii. administering the saliva substitute or saliva stimulant to the subject.

In certain embodiments of both such aspects, step (i) and step (ii) are applied substantially consecutively or, alternatively, the steps are applied concurrently. In certain embodiments, step (i) is performed before step (ii). In certain embodiments, step (ii) is performed before step (i).

In certain embodiments, the signal is applied to preganglionic neurons and/or postganglionic neurons innervating the salivary gland(s), preferably the submandibular gland(s). In certain embodiments, the signal is applied to postganglionic neurons of the CST-SMG axis. In certain embodiments, the signal is applied to postganglionic neurons of the superior cervical ganglion wherein said postganglionic neurons are low-threshold secretomotor neurons.

In certain embodiments, the signal is applied by a neuromodulation apparatus comprising one or more transducers configured to apply the signal. In certain preferred embodiments the neuromodulation apparatus is at least partially implanted in the subject. In certain preferred embodiments, the neuromodulation apparatus is wholly implanted in the subject.

In a further aspect, the invention provides a saliva substitute or saliva stimulant for use in a method of treating xerostomia in a subject, wherein the method comprises administering the saliva substitute or saliva stimulant to the subject, the subject having an apparatus according to the first aspect of the invention implanted such that the neural interfacing element is positioned in signalling contact with a superior cervical ganglion (SCG) of the subject.

In a further aspect the invention provides a saliva substitute or saliva stimulant for use in a method of treating Sjögren's Syndrome in a subject, wherein the method comprises administering the saliva substitute or saliva stimulant to the subject, the subject having an apparatus according the first aspect of the invention implanted such that the neural interfacing element is positioned in signalling contact with a superior cervical ganglion (SCG) of the subject.

The following embodiments relate to equally and independently to those aspects of the invention for use in a method of treating xerostomia or Sjögren's Syndrome in a subject except where indicated otherwise.

In certain embodiments, the treatment of xerostomia is prophylactic treatment. That is, the methods of the invention reduce the likelihood of a subject developing xerostomia, for example if said subject is prescribed a medication known to have xerostomia as a side-effect. The methods of the invention may be used to prevent xerostomia associated with Sjögren's Syndrome in subjects identified as at higher risk of developing this disease as compared with the average population risk.

In certain embodiments, the treatment of xerostomia is therapeutic treatment. That is, the methods of the invention at least partially relieve or ameliorate the severity of xerostomia in subjects exhibiting oral dryness, for example as a side-effect of treatment or as a symptom of another disease, particularly Sjögren's Syndrome. In methods of the invention used to treat xerostomia associated with SS, the alleviation of oral dryness may be accompanied by a decrease in inflammation, particularly oral inflammation.

In certain embodiments, treatment of xerostomia, for example xerostomia associated with Sjögren's Syndrome, is indicated by an improvement in a measurable physiological parameter, for example an improvement in one or more of: systemic sympathetic tone; salivary volume; total protein/peptide concentration of saliva; anti-inflammatory protein/peptide concentration of saliva; secretion from the salivary gland(s); secretion from the submandibular gland(s); levels of oral inflammation; the profile of neural activity in the nerve to which the signal is applied. For example, such an improvement may be a decrease in sympathetic tone; an increase in salivary volume; an increase in the protein/peptide concentration of saliva (for example an increase in total protein concentration and/or an increase in an anti-inflammatory protein or peptide (e.g. SMR1, CABS1, FEG, feG, sialorphin, opiorphin or homologs thereof)); an increase in secretion from the salivary glands; and/or an increase in secretion from the submandibular gland(s). In SS, an improvement in a measurable parameter may be a decrease in inflammation, for example oral inflammation and/or systemic inflammation, optionally accompanied by an improvement in one or more of the parameters recited above.

In certain embodiments, the treatment of Sjögren's Syndrome is prophylactic treatment. That is, the methods of the invention reduce the likelihood of a Sjögren's Syndrome patient developing one or more symptoms of Sjögren's Syndrome, for example xerostomia, ocular dryness, or oral inflammation.

In certain embodiments, the treatment of Sjögren's Syndrome is therapeutic. That is, the methods of the invention at least partially relieve or ameliorate one or more symptoms of Sjögren's Syndrome, for example oral dryness, oral inflammation, systemic inflammation.

In certain embodiments, treatment of Sjögren's Syndrome is indicated by an improvement in a measurable physiological parameter, for example an improvement in one or more of: systemic sympathetic tone; salivary volume; total protein/peptide concentration of saliva; anti-inflammatory protein/peptide concentration of saliva; secretion from the salivary gland(s); secretion from the submandibular gland(s); levels of oral inflammation; the profile of neural activity in the nerve to which the signal is applied. Examples of such improvements in relevant physiological parameters are recited above.

Suitable methods for determining the value for any given parameter will be appreciated by the skilled person.

In certain embodiments, treatment of the condition is indicated by an improvement in the profile of neural activity in the neurons, nerve or nerves to which the signal is applied. That is, treatment of the condition is indicated by the neural activity in the neurons or nerve(s) approaching the neural activity in a healthy individual—i.e. the pattern of action potentials in the nerve more closely resembling that exhibited by a healthy individual than before the intervention.

Stimulation of neural activity as a result of applying the signal is an increase in neural activity in the neurons or nerve to which the signal is applied. In certain embodiments, a signal may be applied to a nerve or nerves resulting in the neural activity in at least some of the neurons (for example, specific classes of neurons) being increased compared to the baseline neural activity in that part of the nerve. Stimulation of neural activity could equally be across the whole nerve, in which case neural activity would be increased for all neurons across the whole nerve or nerves.

For the avoidance of doubt, stimulation of neural activity as used herein is taken to mean a functional increase in signalling activity in the indicated neurons, nerve or nerve fibres. In certain embodiments, the signal is applied to the specified neurons on the left-side of the subject, the specified neurons on the right-side of the subject, or both. That is, in certain embodiments the signal is applied unilaterally or, alternatively, bilaterally.

In certain embodiments, the signal is applied intermittently. In certain such embodiments, the signal is applied for a first time period, then stopped for a second time period, then reapplied for a third time period, then stopped for a fourth time period. In such an embodiment, the first, second, third and fourth periods run sequentially and consecutively. The series of first, second, third and fourth periods amounts to one application cycle. In certain such embodiments, multiple application cycles can run consecutively such that the signal is applied in phases, between which phases no signal is applied.

In certain embodiments, the application cycles are not immediately consecutive. In certain such embodiments the application cycles are separated by a period of 1-60 min, 5-30 min, 10-20 min, optionally 15 min.

In such embodiments, the duration of the first, second, third and fourth time periods is independently selected. That is, the duration of each time period may be the same or different to any of the other time periods. In certain such embodiments, the duration of each of the first, second, third and fourth time periods is any time from 5 seconds (5s) to 24 hours (24h), 30s to 12 h, 1 min to 12 h, 5 min to 8 h, 5 min to 6 h, 10 min to 6 h, 10 min to 4 h, 30 min to 4 h, 1 h to 4 h. In certain embodiments, the duration of each of the first, second, third and fourth time periods is 5s, 10s, 30s, 60s, 2 min, 5 min, 10 min, 20 min, 30 min, 40 min, 50 min, 60 min, 90 min, 2 h, 3 h, 4 h, 5 h, 6 h, 7 h, 8 h, 9 h, 10 h, 11h, 12 h, 13 h, 14 h, 15 h, 16 h, 17 h, 18 h, 19 h, 20 h, 21 h, 22 h, 23 h, 24 h.

In certain embodiments, consecutive application cycles are applied for an operative period—that is, an operative period is a period over which consecutive application cycles are in operation. In such embodiments, the operative period is immediately followed by an inoperative period. In certain embodiments, the operative and inoperative period have a duration independently selected from 1-60 min, 5-30 min, 10-20 min, optionally 15 min. In certain embodiments, the operative and inoperative period have a duration independently selected from 1-24h, 1-12h, 1-6h, optionally 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21h, 22 h, 23 h, 24 h.

In certain embodiments wherein the signal is applied intermittently, the signal is applied for a specific amount of time per day. In certain such embodiments, the signal is applied for 10 min, 20 min, 30 min, 40 min, 50 min, 60 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h per day. In certain such embodiments, the signal is applied continuously for the specified amount of time. In certain alternative such embodiments, the signal may be applied discontinuously across the day, provided the total time of application amounts to the specified time.

In certain embodiments wherein the signal is applied intermittently, the signal is applied only when the subject is in a specific state. In certain such embodiments, the signal is applied only when the subject is in a state of xerostomia. In such embodiments, the status of the subject (e.g. that they are experiencing xerostomia) can be indicated by the subject. In alternative such embodiments, the status of the subject can be detected independently from any input from the subject. In certain embodiments in which the signal is applied by a neuromodulation apparatus, the apparatus further comprises a detector configured to detect the status of the subject, wherein the signal is applied only when the detector detects that the subject is in the specific state.

In certain embodiments of the aspect of the invention relating to a saliva substitute or saliva stimulant for use in a method of treatment comprising the steps of i) applying a signal to a superior cervical ganglion (SCG) of said subject to stimulate neural activity in said SCG; and ii) administering the saliva substitute or saliva stimulant to the subject, the method further comprises the step of detecting one or more physiological parameters of the subject, wherein the signal is applied only when the detected physiological parameter meets or exceeds a predefined threshold value. In such embodiments wherein more than one physiological parameter is detected, the signal may be applied when any one of the detected parameters meets or exceeds its threshold value, alternatively only when all of the detected parameters meet or exceed their threshold values. In certain embodiments wherein the signal is applied by a neuromodulation apparatus, the apparatus further comprises at least one detector element configured to detect the one or more physiological parameters.

In certain embodiments, the one or more detected physiological parameters are selected from: systemic sympathetic tone; salivary volume; total protein/peptide concentration of saliva; anti-inflammatory protein/peptide concentration of saliva; secretion from the salivary gland(s); secretion from the submandibular gland(s).

Similarly, in certain embodiments the detected physiological parameter could be an action potential or pattern of action potentials in postganglionic neurons of the subject, specifically postganglionic neurons of the superior cervical ganglia, wherein the action potential or pattern of action potentials is associated with xerostomia or Sjögren's Syndrome.

It will be appreciated that any two or more of the indicated physiological parameters may be detected in parallel or consecutively. For example, in certain embodiments, the pattern of action potentials in the postganglionic neurons of the superior cervical ganglion can be detected at the same time as secretion by the salivary gland(s), preferably the submandibular gland(s).

In certain embodiments, the signal is permanently applied. That is, once begun, the signal is continuously applied to the neurons, nerve or nerves. It will be appreciated that in embodiments wherein the signal is a series of pulses, gaps between pulses do not mean the signal is not continuously applied.

In certain embodiments, the stimulation in neural activity caused by the application of the signal is temporary. That is, upon cessation of the signal, neural activity in the neurons, nerve or nerves returns substantially towards baseline neural activity within 1-60 seconds, or within 1-60 minutes, or within 1-24 hours, optionally 1-12 hours, optionally 1-6 hours, optionally 1-4 hours, optionally 1-2 hours. In certain such embodiments, the neural activity returns substantially fully to baseline neural activity. That is, the neural activity following cessation of the signal is substantially the same as the neural activity prior to the signal being applied—i.e. prior to modulation.

In certain alternative embodiments, the stimulation of neural activity caused by the application of the signal is substantially persistent. That is, upon cessation of the signal, neural activity in the neurons, nerve or nerves remains substantially the same as when the signal was being applied—i.e. the neural activity during and following stimulation is substantially the same.

In certain embodiments, the stimulation of neural activity caused by the application of the signal is partially corrective, preferably substantially corrective. That is, upon cessation of the signal, neural activity in the neurons, nerve or nerves more closely resembles the pattern of action potentials observed in a healthy subject than prior to stimulation, preferably substantially fully resembles the pattern of action potentials observed in a healthy subject. For example, application of the signal stimulates neural activity, and upon cessation of the signal, the pattern of action potentials in the neurons, nerve or nerves resembles the pattern of action potentials observed in a healthy subject. It is hypothesised that such a corrective effect is the result of a positive feedback loop.

In certain such embodiments, once first applied, the signal may be applied intermittently or permanently, as described in the embodiments above.

In certain embodiments, the signal is applied to postganglionic neurons of one of the superior cervical ganglia. In certain embodiments, the signal selectively stimulates postganglionic neurons of the CST-SMG axis. In certain embodiments, the signal selectively stimulates neural activity in postganglionic neurons of the superior cervical ganglion wherein said postganglionic neurons are low-threshold secretomotor neurons.

Optionally, the signal is applied under regulation at the selection or demand of the subject.

In certain embodiments, the signal is applied bilaterally. That is, in such embodiments, the signal is applied to neurons on both the left and right side of the subject such that neural activity is stimulated in the neurons to which the signal is applied—i.e. the stimulation is bilateral. In such embodiments, the signal applied to right and left SCG, and therefore the extent of stimulation, is independently selected. In certain embodiments the signal applied to the SCG on the right side is the same as the signal applied to the SCG on the left side. In certain alternative embodiments the signal applied to the SCG on the right side is different to the signal applied to the SCG on the left side.

In certain embodiments wherein the modulation is bilateral, each signal is applied by a neuromodulation apparatus comprising one or more transducers for applying the signal. In certain such embodiments, all signals are applied by the same neuromodulation apparatus, that apparatus have at least two transducers, one to apply the signal to the left-side neurons and one to apply the signal to the right-side neurons. In certain alternative embodiments, each signal is applied by a separate neuromodulation apparatus.

In certain embodiments, the signal applied is a non-destructive signal.

In certain embodiments, the signal applied is an electrical signal, an electromagnetic signal (optionally an optical signal), a mechanical (optionally ultrasonic) signal, a thermal signal, a magnetic signal or any other type of signal.

In certain such embodiments in which more than one signal may be applied, for example when the modulation is bilateral, each signal may be independently selected from an electrical signal, an optical signal, an ultrasonic signal, and a thermal signal. In those such embodiments in which two signals are applied by one modulation apparatus, the two signals may be the same type of signal or may be different types of signal independently selected from an electrical signal, an optical signal, an ultrasonic signal, and a thermal signal. In those embodiments in which two signals are applied, each by a separate neuromodulation apparatus, the two signals may be the same type of signal or may be different types of signal independently selected from an electrical signal, an optical signal, an ultrasonic signal, and a thermal signal.

In certain embodiments in which the signal is applied by a neuromodulation apparatus comprising at least one transducer, the transducer may be comprised of one or more electrodes, one or more photon sources, one or more ultrasound transducers, one more sources of heat, or one or more other types of transducer arranged to put the signal into effect.

In certain embodiments, the signal is an electrical signal, for example a voltage or current, and the transducer is an electrode, for example a wire electrode or a cuff electrode. In certain such embodiments the signal comprises a direct current (DC) waveform, such as a charge balanced DC waveform, or an alternating current (AC) waveform, or both a DC and an AC waveform.

In certain embodiments, the DC waveform or AC waveform may be a square, sinusoidal, triangular or complex waveform. The DC waveform may alternatively be a constant amplitude waveform. In certain embodiments the electrical signal is a DC square waveform of varying voltage.

In certain embodiments, the electrical signal is a DC or AC waveform having a frequency in the range of 1 Hz-1 kHz, optionally 1-500 Hz, optionally 1-200 Hz, 1-100 Hz, 1-50 Hz, 1-20 Hz, 1-10 Hz, 5-10 Hz, optionally 5 Hz or 7.5 Hz, optionally 50-150 Hz, optionally 100 Hz, or within any interval between the described upper and lower limits.

In certain embodiments wherein the signal is an electrical signal, the electrical signal has a pulse duration of 0.001-5 ms, 0.01-5 ms, 0.1-5 ms, 1-5 ms, 1-2 ms, optionally 2 ms. In certain embodiments the signal has a pulse duration of 0.005-0.1 msec, optionally 0.01-0.06 ms. optionally 0.01-0.05 msec, optionally 0.01-0.04 ms. In certain preferred embodiments the signal has a pulse duration of 0.01-0.03 msec, more preferably 0.01-0.02 msec.

In certain embodiments wherein the signal is an electrical signal the signal has a pulse duration of less than or equal to 0.1 msec, optionally less than or equal to 0.06 msec, optionally less than or equal to 0.05 msec, optionally less than or equal to 0.04 msec, optionally less than or equal to 0.03 msec, optionally less than or equal to 0.02 msec, optionally less than or equal to 0.01 ms. In certain preferred embodiments the signal has a pulse duration of 0.01 msec or 0.02 msec or 0.04 msec.

In certain embodiments, the electrical signal comprises a DC waveform and/or an AC waveform having a current of 10-2000 μA, optionally 20-1000 μA, 50-1000 μA, 100-1000 μA, 500-1000 μA, 500-800 μA, optionally 800 μA. In certain embodiments the signal has a current of 20-500 μA, optionally 50-250 μA. In certain embodiments the electrical signal has a current of at least 10 μA, at least 20 μA, at least 50 μA, at least 60 μA, at least 70 μA, at least 80 μA, at least 90 μA, at least 100 μA, at least 110 μA, at least 150 μA, at least 180 μA, at least 200 μA, at least 220 μA, at least 250 μA, at least 300 μA, at least 400 μA, at least 500 μA, at least 600 μA, at least 700 μA, at least 800 μA. These ranges are illustrative, and one of skill in the art will recognize that the lower and upper limits can vary independently.

In certain preferred embodiments of all aspects, the signal comprises a DC or AC square waveform of 5 Hz-7.5 Hz, pulse duration 2 msec, current 0.8 mA.

It will be appreciated by the skilled person that the current amplitude of an applied electrical signal necessary to achieve the intended stimulation will depend upon the positioning of the electrode and the associated electrophysiological characteristics (e.g. impedance). It is within the ability of the skilled person to determine the appropriate current amplitude for achieving the intended stimulation in a given subject. For example, the skilled person is aware of methods suitable to monitor the neural activity profile induced by nerve stimulation.

In another aspect, the invention provides a neuromodulatory electrical waveform for use in treating xerostomia, for example xerostomia associated with Sjögren's Syndrome, in a subject, wherein the waveform is an alternating current or direct current (DC) waveform having a frequency of 1-1000 Hz, such that, when applied to neurons of a superior cervical ganglion of the subject, the waveform stimulates neural signalling in the neurons, preferably selectively stimulating neural activity in the postganglionic neurons innervating the submandibular gland. In certain embodiments, the waveform, when applied to the neurons, relieves or prevents xerostomia.

In another aspect, the invention provides a neuromodulatory electrical waveform for use in treating Sjögren's Syndrome in a subject, wherein the waveform is an alternating current or direct current (DC) waveform having a frequency of 1-1000 Hz, such that, when applied to neurons of a superior cervical ganglion of the subject, the waveform stimulates neural signalling in the neurons, preferably selectively stimulating neural activity in the postganglionic neurons innervating the submandibular gland. In certain embodiments, the waveform, when applied to the neurons, relieves or prevents xerostomia.

In another aspect, the invention provides use of a neuromodulation apparatus for treating xerostomia, in particular xerostomia associated Sjögren's Syndrome, in a subject by stimulating neural activity in neurons of a superior cervical ganglion of the subject, preferably postganglionic neurons innervating the submandibular gland.

In another aspect, the invention provides use of a neuromodulation apparatus for treating Sjögren's Syndrome, in particular xerostomia associated Sjögren's Syndrome, in a subject by stimulating neural activity in neurons of a superior cervical ganglion of the subject, preferably postganglionic neurons innervating the submandibular gland.

In a preferred embodiment of all aspects of the invention, the subject or patient is a mammal, more preferably a human, such as a patient experiencing xerostomia (e.g., a patient with Sjögren's Syndrome) or a patient having Sjögren's Syndrome.

In a preferred embodiment of all aspects of the invention, the signal or signals is/are applied substantially exclusively to the neurons or nerves specified, and not to other neurons or nerves.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

### EXAMPLES

### Example 1

Experiments were performed to establish the roles of (1) the cervical sympathetic chains (CSC), which contain the pre-ganglionic nerve fibers that innervate the post-ganglionic cell bodies located in the superior cervical ganglion (SCG), and (2) post-ganglionic trunks of the SCG, namely the internal (ICN) and external (ECN) carotid nerves, on regulating arterial blood flow and vascular resistances within the submandibular glands (SMG) of rats and mice. This involves monitoring the changes in blood flow in the left and right SMG by laser doppler and electromagnetic flow probes elicited by (1) direct electrical stimulation of the ipsilateral CSC, ICN and ECN, (2) the transection of the ipsilateral CSC, ICN and ECN, and (3) physiological activation of CSC-SCG input to the SMG by nociceptive challenge, and by hypoxic-hypercapnic gas (10% O₂, 5% CO₂, 85% N₂) challenges to mimic changes in arterial blood chemistry elicited by episodes of apnea, before and following bilateral transection of the CSC, ICN or ECN.

Experimental Details in a Preliminary Study in Urethane-Anesthetized Mice

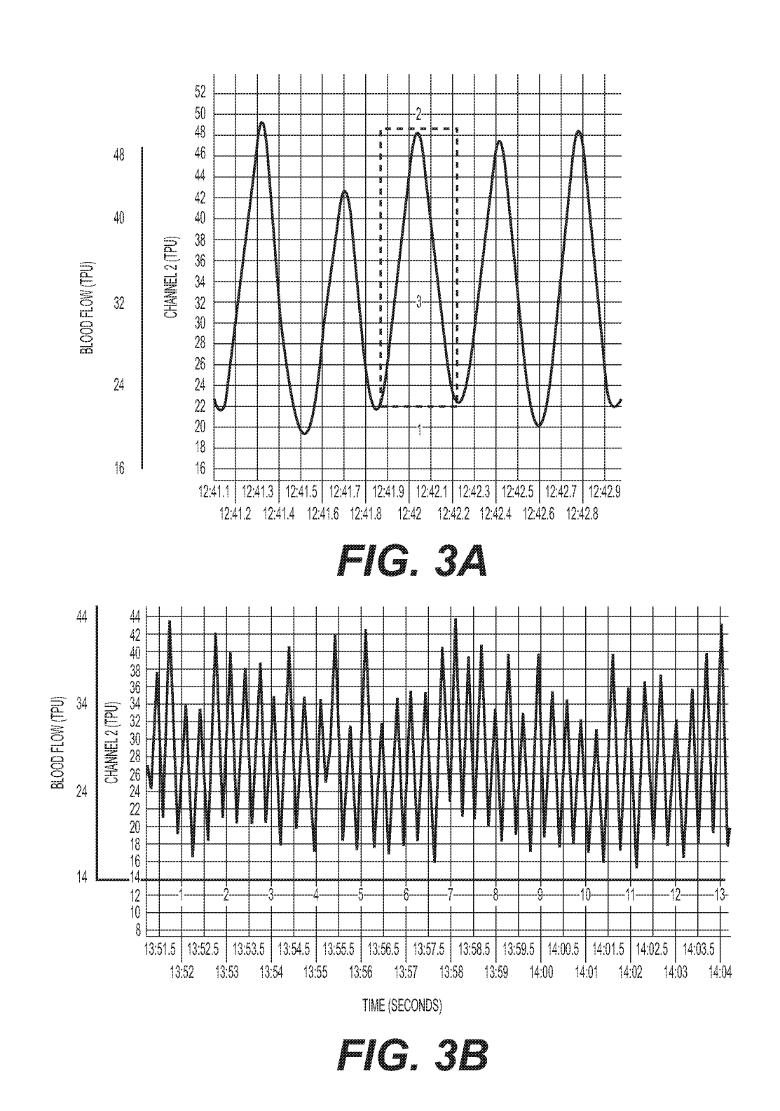

Studies were performed in urethane-anesthetized C57BL6 mice. A Laser Doppler was placed in direct contact with the left submandibular gland (SMG). Paw-pinch was applied to activate sympathetic nerves and especially the cervical sympathetic chain-superior cervical ganglion (CSC-SCG, also known as the CST-SCG) input to SMG. Upper airway occlusion was performed in order to activate cardiopulmonary afferent-mediated effects on SMG blood flow. Finally, CSCs were electrically stimulated via placement of bipolar electrodes directly of the chain. FIG. 3 shows a schematic of the laser Doppler signal (FIG. 3A) and the pattern of signals observed in mice. The heart rates of urethane-anesthetized mice are about 600 beats/min. The laser Doppler pulses are 3.8±0.6 pulses/sec=228±36 pulses/min, which are in line with average breaths/min for these mice (FIG. 3B)

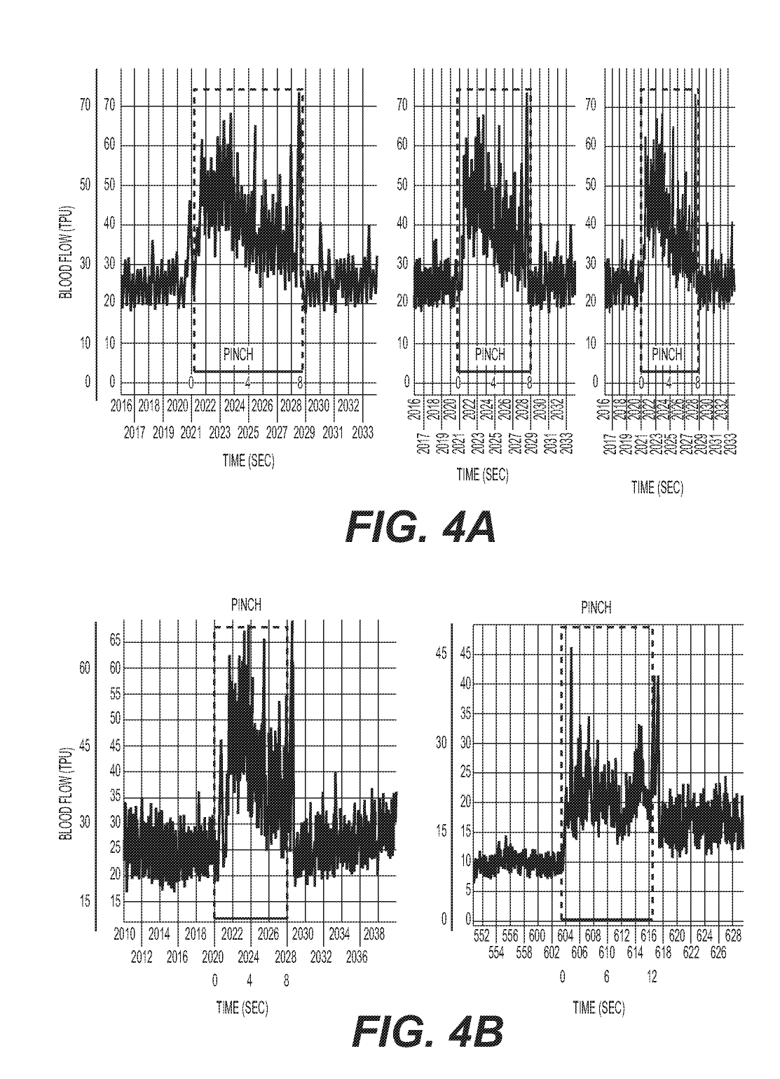

Pinching the back paw (6s) elicited a notable increase in blood flow within the SMG, as shown in FIG. 4A (note that the same response is shown in each panel, just in different time scales).

Unlike shorter episodes of paw-pinch, cessation of a 12 second episode was not associated with an immediate recovery of blood flow (FIG. 4B).

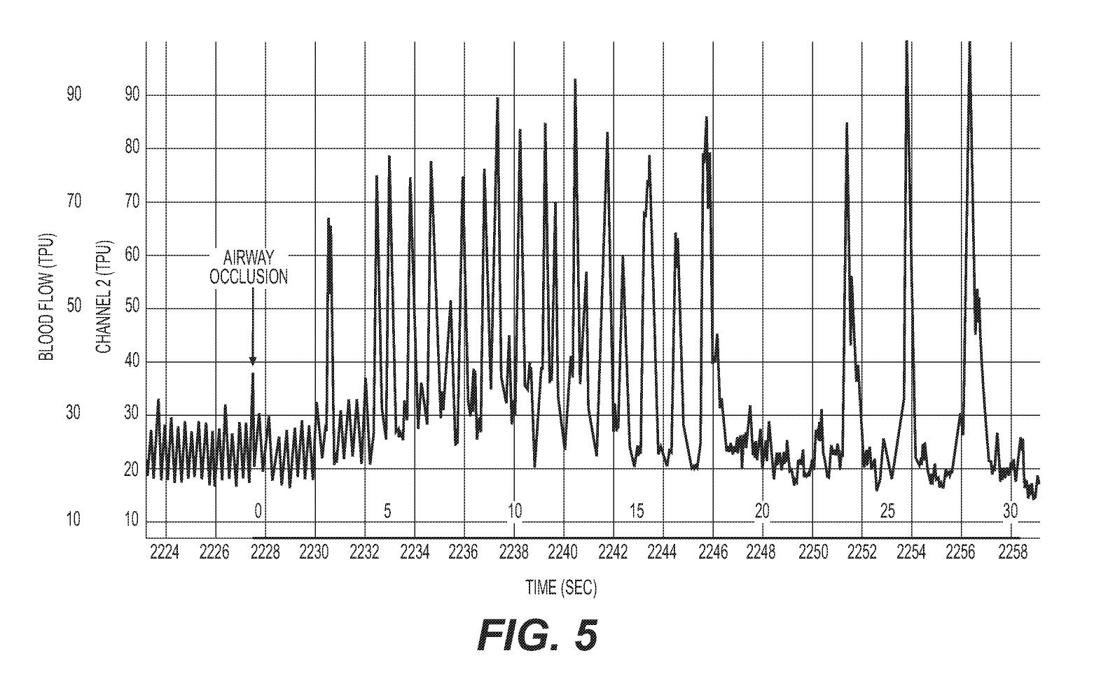

Airway occlusion also increases blood flow in the SMG. This can be seen in the period from 5-20 seconds in FIG. 5.

Electrical Stimulation

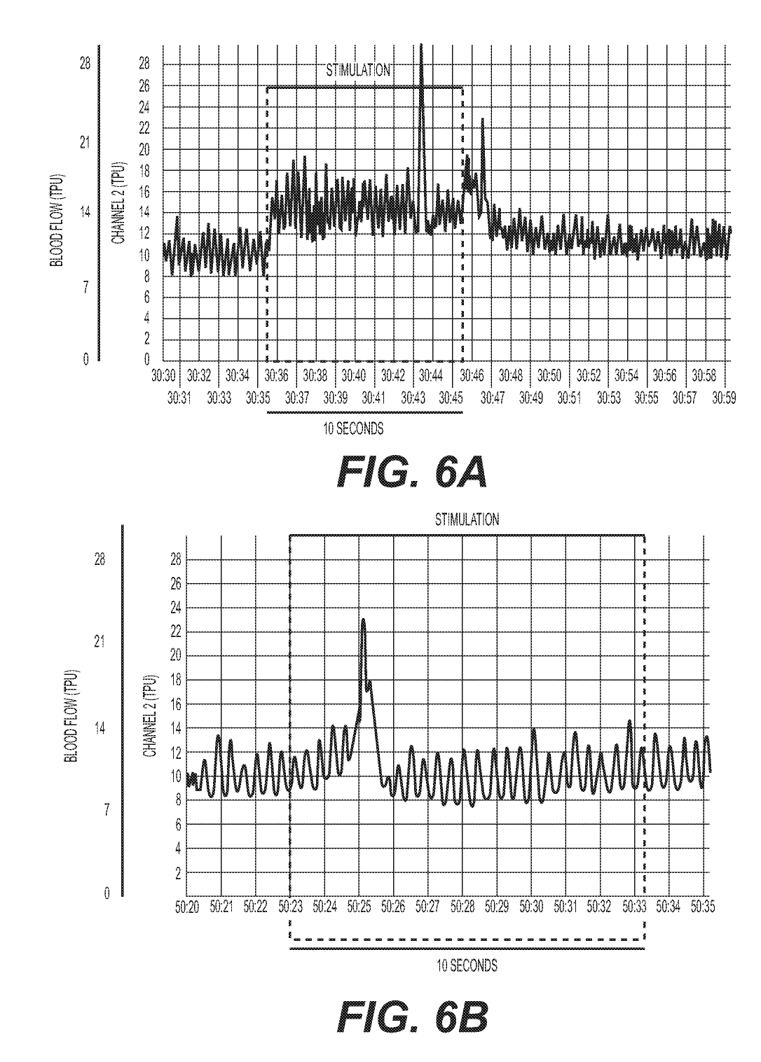

Electrical stimulation (0.8 mA, 5 Hz) of the right CSC increases blood flow in the right SMG (FIG. 6A). The increase in blood flow suggests active neurogenic vasodilation as result of the stimulation. Transection of the ipsilateral (right) ICN and ECN markedly diminishes the response elicited by electrical stimulation of the right CSC (0.8 mA, 5 Hz) (FIG. 6B).

Summary

These data demonstrate that blood flow in the microvasculature of the mouse SMG is under respiratory modulation and responds to a nociceptive challenge and occlusion of the airway. The abrupt beginning and end to the changes in flow associated with start and end of challenge makes it likely that the changes are due to alterations in CSC-SCG input.

In addition, blood flow in the mouse SMG responds to electrical stimulation of the CSC. These responses are largely eliminated (−86+7.9%, n=8) by combined transection of the ipsilateral ICN and ECN.

### Example 2

Levels of the anti-inflammatory pro-hormone SMR1 (FIG. 7A) in rat SMG were measured by Western Blot. The resulting data (FIG. 7B) corresponded to previously reported data (Morris et al, supra).

Electrical Stimulation

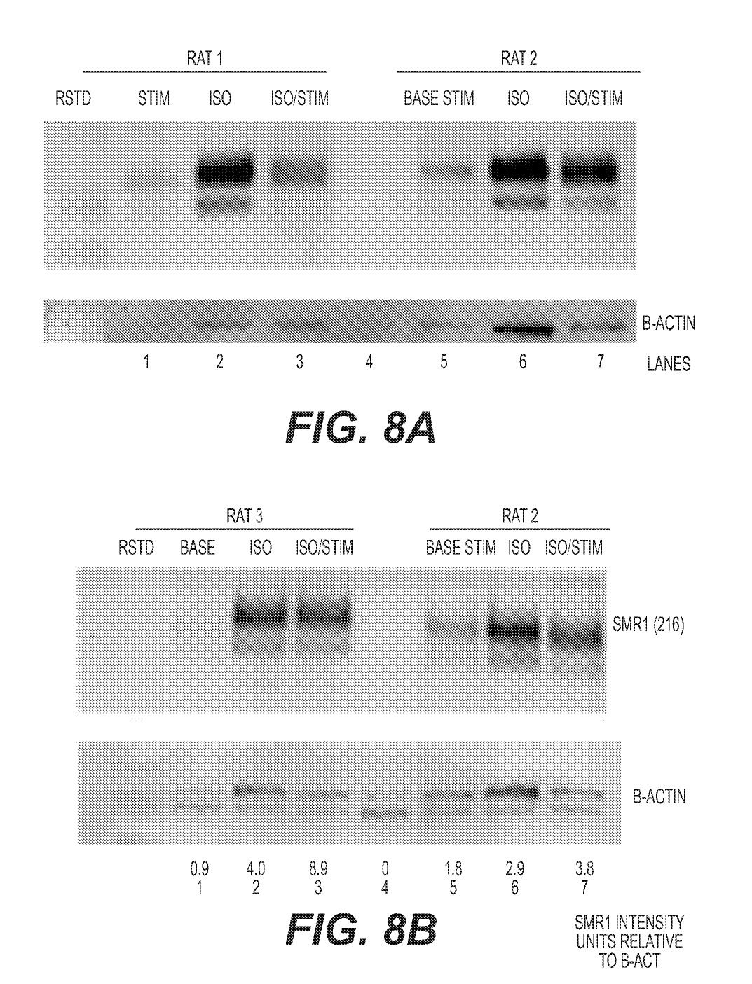

The CSCs of 3 male rats were stimulated with a signal of 0.8 mA 7.5 Hz 2 ms for 30 seconds. Rats were also administered the β-adrenoreceptor agonist isoproterenol (25 mg/kg i.p). Saliva was collected at baseline prior to stimulation, during stimulation, following isoproterenol administration, and after both isoproterenol administration and subsequent electrical stimulation. The level of SMR1 in the collected saliva was measured by Western blot (equal total protein load per lane). The results for rat #1 and rat #2 are shown in FIG. 8A (no measurement was collected for saliva prior to stimulation in rat #1). The results for rat #3 and rat #2 are shown in FIG. 8B, with relative SMR1 amounts shown below.

As can be seen, electrical stimulation increased SMR1 levels in saliva compared to baseline levels before stimulation (FIG. 8). In addition, electrical stimulation was able to further increase levels of SMR1 in rats that had already received sympathetic agonist treatment (iso vs iso/stim in FIG. 8). These data indicate that electrical stimulation is effective at increasing SMR1 production in saliva.

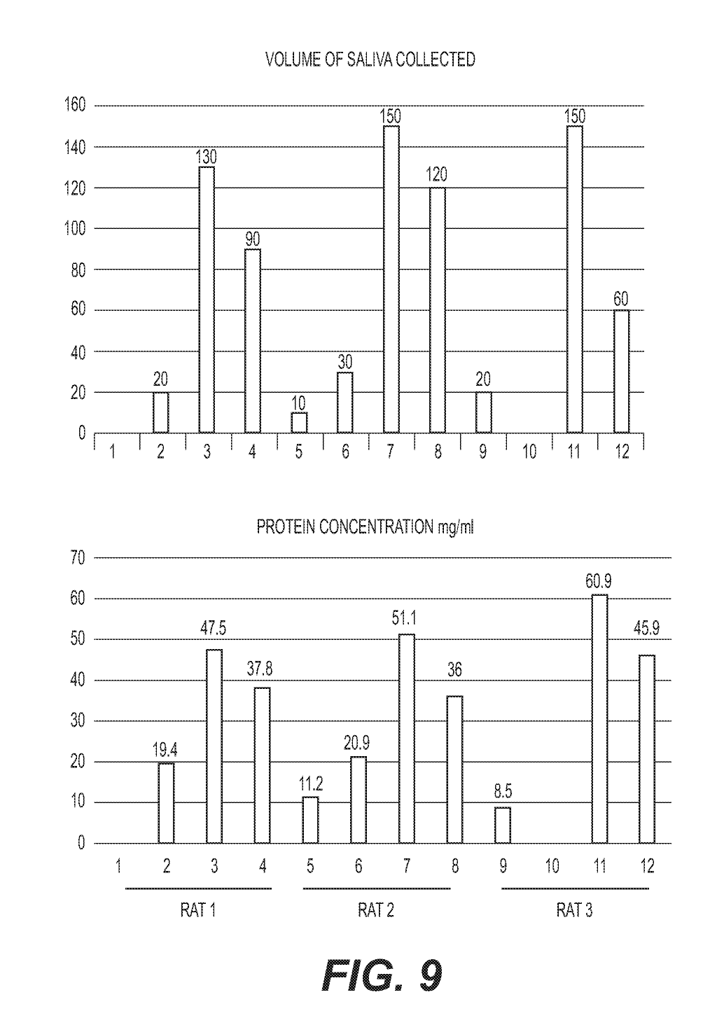

Saliva volume was also collected from the 3 rats. FIG. 9 demonstrates electrical stimulation can also increase the volume of saliva collected and the total content of protein in the collected saliva. Numbers 1, 5 and 9 are prestimulation samples, 2, 6 and 10 post-stimulation, 3, 7 and 11 post-isoproterenol, and 4, 8 and 12 post-stimulation and isoproterenol for rats #1, #2, and #3 respectively.

An additional experiment was performed using 2 Sprague-Dawley male rats (12 weeks of age) to confirm the effect of electrical stimulation on saliva production. Cervical sympathetic chain (CSC) proximal to the SCG was electrically stimulated, using 0.8 mA, 7.5 Hz, 2 ms, 30 sec on, 30s off for 15 min. Each rat got four stimulations total, two on the left and two on the right side. Saliva was collected for 30 min prior to stimulation (#1, #9), for 20 min during stimulation (even numbers), and 15-20 min between stimulation (odd numbers) into tubes containing inhibitors. Protein concentration was determined by Bicinchoninic Acid (BCA) protein assay. Western blots were probed with SMR1(216).

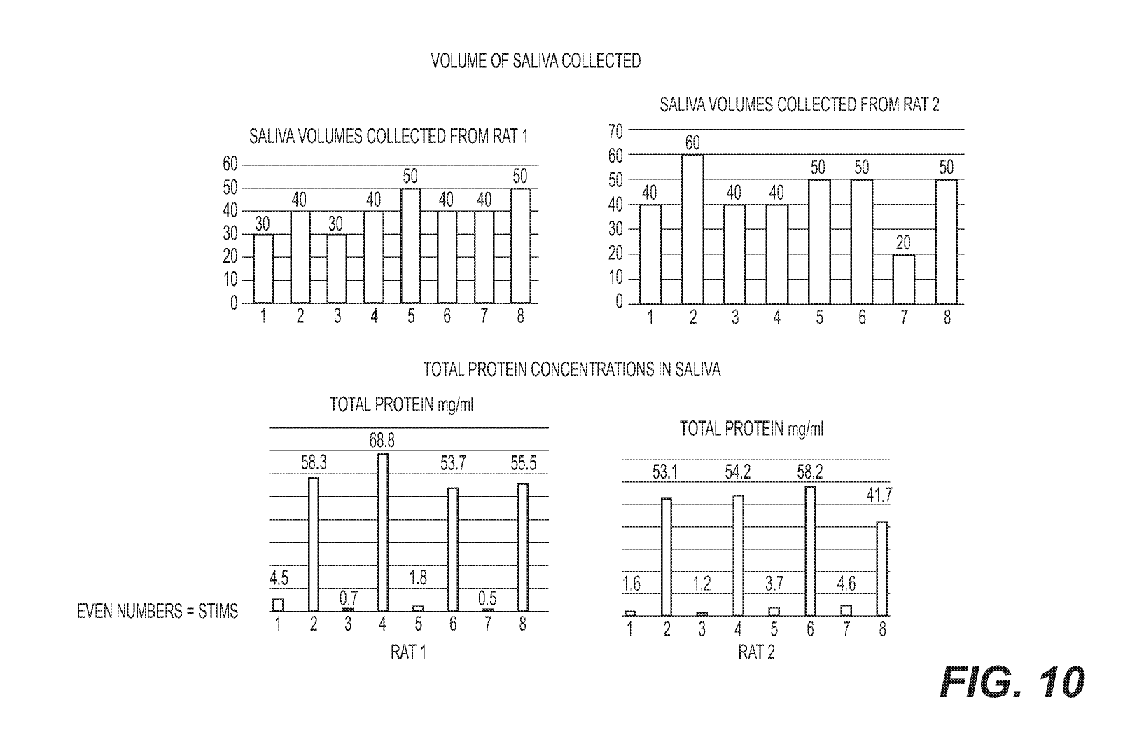

Total saliva volume collected at each point and total protein concentration in the collected saliva is shown in FIG. 10 (top 2 panels and bottom 2 panels, respectively). The first stimulation in each rat resulted in an increase in saliva volume during stimulation. In both rats, the intermediate stimulations had variable effects on saliva volume. Without wishing to be bound by theory, this may be due to the secretory glands not having the opportunity to fully refill between stimulations.

Nevertheless, at the end of the stimulation cycles, both rats exhibited higher saliva volumes collected compared to each rat prior to any stimulation (column 1 vs column 8 in FIG. 10, top 2 panels).

Total protein concentration in the collected saliva was also determined (FIG. 10, bottom panels). In both rats, saliva collected during stimulation had dramatically increased levels of protein (mg/ml).

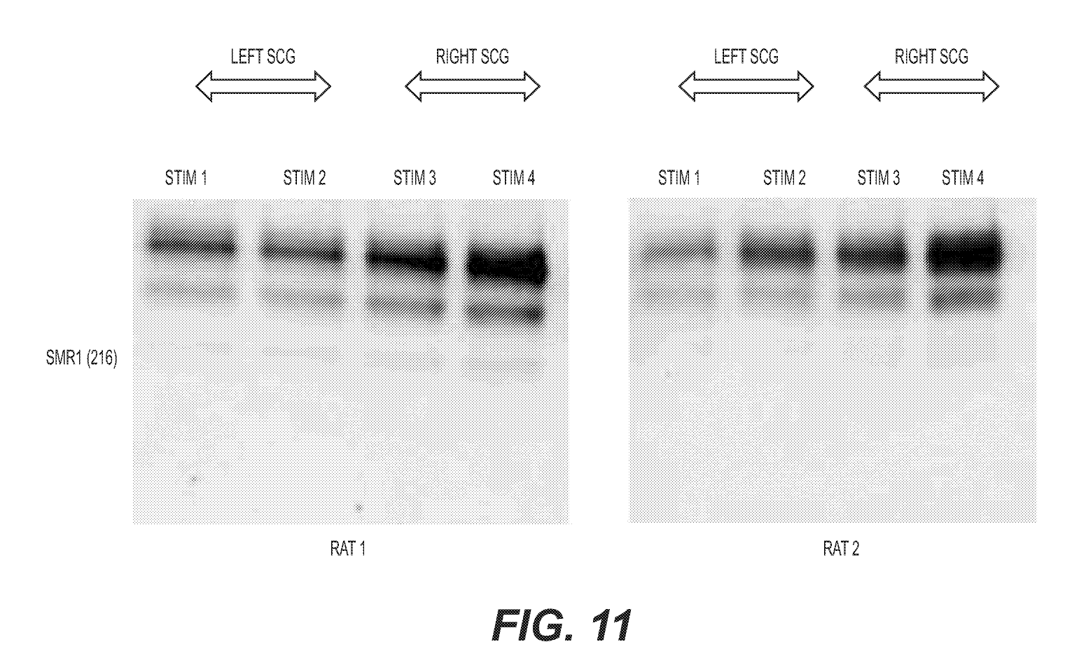

Following each stimulation levels of SMR1 in saliva also increased. FIG. 11 shows Western blots of saliva taken during each stimulation for both rat 1 and rat 2 (equal total protein loaded per lane). Each stimulation results in successive increases in levels of SMR1. This is in addition to the increase in total protein content shown in FIG. 10, as the Western blots were loaded with equal total protein. Thus CSC electrical stimulation increases total protein content and levels of the anti-inflammatory peptide SMR1 in addition to increasing production of saliva.

1.-61. (canceled)

62. A apparatus for increasing at least one of saliva production from a baseline and anti-inflammatory peptide in saliva from a baseline of a subject, the apparatus comprising:
one or more neural interfacing elements comprising a transducer, each positioned to apply a signal to a superior cervical ganglion (SCG) of the subject; and
a controller operably coupled to the one or more neural interfacing elements, the controller configured to increase at least one of the saliva production from the baseline and the anti-inflammatory peptide in the saliva from the baseline by controlling the signal to be applied by each of the one or more neural interfacing elements.

63. The apparatus of claim 62, wherein the increase in at least one of the saliva production from the baseline and the anti-inflammatory peptide in the saliva from the baseline treats xerostomia.

64. The apparatus of claim 62, wherein the increase in at least one of the saliva production from the baseline and the anti-inflammatory peptide in the saliva from the baseline treats Sjögren's Syndrome.

65. The apparatus of claim 62, wherein the one or more neural interfacing elements is further positioned to apply a signal to at least one of a preganglionic and a postganglionic neuron of the SCG.

66. The apparatus of claim 62, wherein the signal selectively stimulates neural activity in neuron innervating at least one of salivary glands.

67. The apparatus of claim 66, wherein the increase in at least one of the saliva production from the baseline and the anti-inflammatory peptide in the saliva from the baseline comprises one or more of: an increase in saliva volume; an increase in saliva secretion from the salivary glands; an increase in saliva secretion from submandibular glands; a decrease in apparatusic inflammation; a decrease in oral inflammation; an increase in anti-inflammatory peptide secretion; an increase in anti-inflammatory peptide expression by the submandibular glands; an increase in production of SM R1 or a human homolog thereof and/or an increase in peptides derived therefrom; an increase in production of CABS1, opiorphin and/or an increase in peptides derived therefrom.

68. The apparatus of claim 62, wherein one or more physiological parameters in the subject is detected and wherein the signal is applied only when a detected one or more physiological parameter meets or exceeds a respective threshold value, each of the one or more physiological parameters has a respective threshold value.

69. The apparatus of claim 68, wherein the one or more detected physiological parameters is selected from apparatusic sympathetic tone; salivary volume; total protein/peptide concentration of saliva; anti-inflammatory protein/peptide concentration of saliva; secretion from salivary glands; and secretion from submandibular glands.

70. The apparatus of claim 62, wherein the one or more neural interference elements comprises two or more neural interference elements bilaterally positioned to apply a signal to a left superior cervical ganglion (SCG) and a right superior cervical ganglion (SCG).

71. The apparatus of claim 62, wherein at least the one or more neural interfacing elements are implantable around the SCG.

72. The apparatus of claim 62, wherein the controller is implantable.

73. The apparatus of claim 62, further comprising a saliva substitute or saliva stimulant.

74. The apparatus of claim 62, wherein the stimulation in neural activity is temporary.

75. The apparatus of claim 62, further comprises an interface for receiving control input from the subject, wherein the controller is configured to apply the signal based on the control input.

76. The apparatus of claim 71, wherein a respective neural interface element of the one or more interfacing elements and the controller is mounted in a housing of a neural modulation device, the neural modulation device further comprises a communication interface configured to communicate externally from the subject.

77. The apparatus of claim 76, wherein the controller is configured to applied the signal based on a control signal received by the communication interface.

78. A method for increasing at least one of saliva production from a baseline and anti-inflammatory peptide in saliva from a baseline of a subject, the method comprising:
implanting in the subject one or more neural interfacing elements comprising a transducer;
positioning the transducer in signaling contact with a superior cervical ganglion (SCG) of the subject; and
increasing at least one of the saliva production from the baseline and the anti-inflammatory peptide in the saliva from the baseline by controlling the signal to be applied by each of the one or more neural interfacing elements.

79. The method of claim 78, wherein the increase in at least one of saliva production from a baseline and anti-inflammatory peptide in the saliva from a baseline treats at least one of xerostomia and Sjögren's Syndrome.
